(19) United States
(12) Patent Application Publication
(10) Pub. No.: US 2024/0271176 A1
(43) Pub. Date: Aug. 15, 2024

(54) POLYMERIC ORGANOMETALLIC REDOX MEDIATOR FOR CONTINUOUS KETONE AND GLUCOSE MONITORING

(71) Applicant: Evolve Scientific Solutions LLC, Milpitas, CA (US)

(72) Inventors: Thulasidharan Gandhi, Carlsbad, CA (US); Anthony Yesudass S, Tamil Nadu (IN)

(21) Appl. No.: 18/106,252

(22) Filed: Feb. 6, 2023

(51) Int. Cl. C12Q 1/00; G01N 33/66

(52) U.S. Cl. CPC C12Q 1/004; G01N 33/66

(57) ABSTRACT

Organometallic redox mediator compounds, such as substituted ferrocenes or other metallocenes, and redox polymers comprising the organometallic redox mediators are described. The organic groups of the organometallic redox mediators can be substituted with multiple (e.g., at least three or at least four) electron-donating substituents, which can reduce the redox potential of the redox mediator compared to the corresponding unsubstituted redox mediator. Electrodes coated with the redox polymers or blends comprising the redox polymers are described, as are related electrochemical sensors. In addition, methods of using the sensors to detect biological analytes of interest, such as glucose or ketones, are described.

### TECHNICAL FIELD

The presently disclosed subject matter relates to polymers comprising organometallic (e.g., metallocene-based) redox mediators. The organometallic redox mediators comprise organic moieties substituted with a plurality of electron-donating substituents. The polymers can be used to prepare electrode coatings for electrodes for use in electrochemical sensors for detecting analytes, including biological analytes, such as glucose and ketones. The presently disclosed subject matter further relates to redox mediator-containing monomers that can be used to prepare the polymers, to polymer coated electrodes, and to related electrochemical sensors.

### BACKGROUND

Diabetes is a chronic disease that can lead to a number of serious complications, such as heart disease, kidney failure, and blindness. Continuous monitoring of blood glucose levels can help prevent diabetic patients from developing these complications and can also be useful in the early diagnosis of diabetes. In addition, Continuous Glucose Monitoring (CGM) technologies can be used by doctors to diagnose and treat patients remotely. Diabetic Ketoacidosis (DKA) is a severe complication in Type 1 diabetic patients due to insulin deficiency, which leads to increased ketones (e.g., 3-hydroxybutyrate, acetone, and acetoacetic acid) in the blood, and can result in diabetic coma or death. Therefore, determination and/or monitoring of ketone levels (e.g., hydroxybutyrate levels) in diabetic patents is also desirable for early diagnosis of ketonaemia.

In CGM technologies, patient glucose levels (e.g., blood glucose levels) can be monitored essentially in real time by electrochemical glucose sensors. The sensors can include an enzyme that reacts with glucose. For instance, the enzyme glucose oxidase (GO) oxidizes glucose to produce glucanolactone and hydrogen peroxide (H₂O₂). Thus, the change in H₂O₂ can be monitored to determine glucose concentration because, for each glucose molecule metabolized, there is a proportional change in the product H₂O₂. First generation electrochemical sensors involving GO were developed to monitor H₂O₂ via an oxidation reaction of the H₂O₂ at the surface of a working electrode that produces two protons, two electrons, and one molecule of oxygen. However, the measurement of H₂O₂ involves a high potential range for selectivity as well as on the controlled solubility of oxygen in biological fluids to act as an electron acceptor.

To overcome the dependence on tissue oxygen, second-generation electrochemical sensors have been developed based on mediated electron transfer techniques. Electron transfer redox mediators included in the sensors are considered as the electron acceptors and the reduced redox mediators are measured by the electrodes. The redox mediators can be organic, inorganic, or metal-organic materials. Thus, in these second-generation sensors, redox mediators shuttle electrons and assist in electrical communication between an enzyme and the working electrode. A variety of redox mediators can be used, including organic compounds, inorganic compounds, and metal-organic compounds.

However, there remains an ongoing need for additional redox active materials for use in electrochemical sensors. In particular, there is an ongoing need for redox mediators with tailorable reduction potential for use in detecting analytes in complex samples, such as biological samples, that can include other compounds that can permeate active electrode surfaces and interfere with analyte signal.

### SUMMARY

This summary lists several embodiments of the presently disclosed subject matter, and in many cases lists variations and permutations of these embodiments. This summary is merely exemplary of the numerous and varied embodiments. Mention of one or more representative features of a given embodiment is likewise exemplary. Such an embodiment can typically exist with or without the feature(s) mentioned, likewise, those features can be applied to other embodiments of the presently disclosed subject matter, whether listed in this summary or not. To avoid excessive repetition, this summary does not list or suggest all possible combinations of such features.

In some embodiments, the presently disclosed subject matter provides a redox polymer comprising: at least one polymeric chain; and a plurality of metallocene groups, wherein each of the plurality of metallocene groups comprises a metal atom bound to two arene groups, wherein the two arene groups are together substituted by at least three electron-donating substituents and one substituent that is covalently or non-covalently bound to the at least one polymeric chain. In some embodiments, each of the two arene groups is a cyclopentadienyl group. In some embodiments, the metal atom is an atom of an element selected from the group comprising Fe, Ru, Mn, Os, V, Co, Sc, Ti, Cr, Cu, Zn, Ni, Mo, Rh, Pd, Cd, Pt, and Ir, optionally wherein M is a Fe atom. In some embodiments, the redox potential of each of the plurality of metallocene groups is less than about 0.2 volts (V) versus a silver/silver chloride (Ag/AgCl) reference electrode, optionally wherein the redox potential of each of the plurality of metallocene groups is less than about 0.1 V versus a Ag/AgCl reference electrode.

In some embodiments, each of the plurality of metallocene groups has a structure of formula (I):

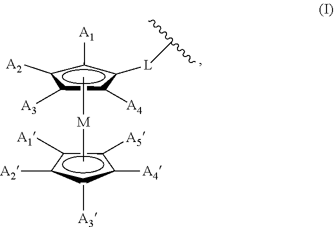

wherein: M is a metal atom of an element selected from the group comprising Fe, Ru, Mn, Os, V, Co, Sc, Ti, Cr, Cu, Zn, Ni, Mo, Rh, Pd, Cd, Pt, and Ir, optionally Fe; L is a linking group that is covalently bonded to the at least one polymeric chain; A₁, A₂, A₃, and A₄ are each independently H or E, subject to the proviso that at least one of A₁, A₂, A₃, and A₄ is E; A₁′, A₂′, A₃′, A₄′, and A₅′ are each independently H or E, wherein at least two of A₁′, A₂′, A₃′, A₄′, and A₅′ are E; and each E is an electron-donating substituent. In some embodiments, each E is independently selected from the group comprising alkyl, alkenyl, alkynyl, aralkyl, aryl, —N(R)₂, —NHR, —NH₂, —OH, —OR, —NHC(═O)R, and —OC(═O)R, wherein each R is independently selected from alkyl, aralkyl, and aryl.

In some embodiments, at least three of A₁, A₂, A₃, and A₄ are E and at least four of A₁′, A₂′, A₃′, A₄′, and A₅′ are E. In some embodiments, each of A₁, A₂, A₃, A₄, A₁′, A₂′, A₃′, A₄′, and A₅′ is E. In some embodiments, each E is C1-C10 alkyl. In some embodiments, each E is methyl.

In some embodiments, L is selected from the group comprising:

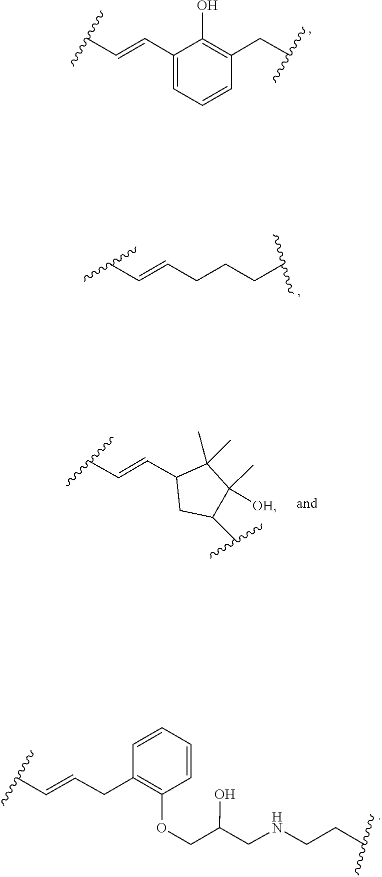 [SMILES: */C=C/c1cccc(C*)c1O ; */C=C/CCC* ; */C=C/C1CC(*)C(C)(O)C1(C)C ; */C=C/Cc1ccccc1OCC(O)CNCC*]

In some embodiments, the redox polymer is a homopolymer, optionally a homopolymer prepared by polymerization of a metallocene-containing monomer having a structure of formula (I′):

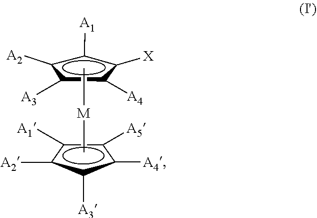

wherein: M is an atom of an element selected from the group comprising Fe, Ru, Mn, Os, V, Co, Sc, Ti, Cr, Cu, Zn, Ni, Mo, Rh, Pd, Cd, Pt, and Ir, optionally Fe; X is a substituent that comprises one or more reactive functional groups selected from the group comprising vinyl, alkenyl, OH, amino, epoxy, carboxylic acid, and isocyanate; A₁, A₂, A₃, and A₄ are each independently H or E, subject to the proviso that at least one of A₁, A₂, A₃, and A₄ is E; A₁′, A₂′, A₃′, A₄′, and A₅′ are each independently H or E, wherein at least two of A₁′, A₂′, A₃′, A₄′, and A₅′ are E; and each E is an electron-donating aryl group substituent. In some embodiments, the redox polymer has a structure of the formula:

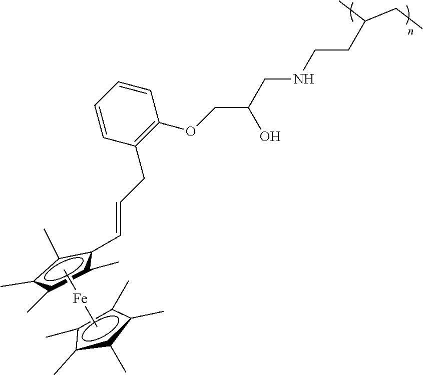

where n is an integer greater than 1.

In some embodiments, the redox polymer is a copolymer prepared by copolymerization of at least two different monomers, wherein one of the monomers is a metallocene-containing monomer having a structure of formula (I′):

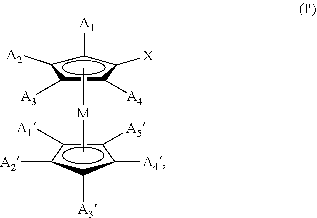

wherein: M is an atom of an element selected from the group comprising Fe, Ru, Mn, Os, V, Co, Sc, Ti, Cr, Cu, Zn, Ni, Mo, Rh, Pd, Cd, Pt, and Ir, optionally Fe; X is a substituent that comprises one or more reactive functional groups selected from the group comprising vinyl, alkenyl, OH, amino, epoxy, carboxylic acid, and isocyanate; A₁, A₂, A₃, and A₄ are each independently H or E, subject to the proviso that at least one of A₁, A₂, A₃, and A₄ is E; A₁′, A₂′, A₃′, A₄′, and A₅′ are each independently H or E, wherein at least two of A₁′, A₂′, A₃′, A₄′, and A₅′ are E; and E is an electron-donating aryl group substituent; optionally wherein the copolymer is a random copolymer, a block copolymer and a graft copolymer.

In some embodiments, at least one of the at least two different monomers is a water-soluble acrylic monomer or a zwitterionic monomer. In some embodiments, the copolymer is a terpolymer prepared by copolymerization of (i) a metallocene-containing monomer having a structure of formula (I′), (ii) a zwitterionic monomer, and (iii) a third monomer, optionally a water soluble acrylic monomer. In some embodiments, the copolymer has a structure of formula (III):

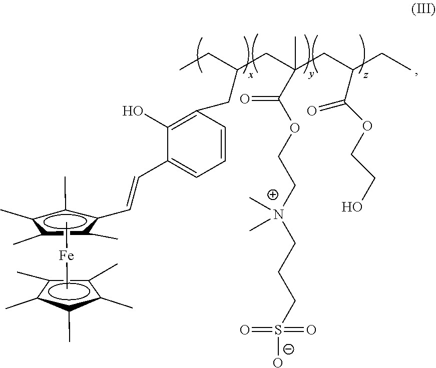

where x, y, and z are each integers, optionally wherein each of x, y, and z are integers greater than 1, further optionally wherein each of x, y, and z are integers greater than 10.

In some embodiments, the presently disclosed subject matter provides a blend comprising a redox polymer of the presently disclosed subject matter and one or more additional polymers; optionally wherein the one or more additional polymers are water soluble.

In some embodiments, the presently disclosed subject matter provides an electrode coated with a coating comprising a redox polymer of the presently disclosed subject matter or a blend thereof. In some embodiments, the coating has a thickness of about 1 μmicrometer (μm) to about 20 μm, optionally about 2 μm to about 5 μm. In some embodiments, the coating further comprises an enzyme, optionally an oxidase or a dehydrogenase, further optionally wherein the enzyme is selected from glucose oxidase, glucose dehydrogenase, and 3-hydroxybutyrate dehydrogenase. In some embodiments, the redox polymer is crosslinked. In some embodiments, the electrode further comprises an outer membrane over the coating, wherein the outer membrane is a semi-permable membrane, optionally wherein the semi-permeable membrane is a polyurethane or silicone-based membrane.

In some embodiments, the presently disclosed subject matter provides a sensor for detecting an analyte of interest, wherein the sensor comprises a working electrode, wherein the working electrode is an electrode coated with a coating comprising a redox polymer of the presently disclosed subject matter or a blend thereof, optionally wherein the analyte of interest comprises a plurality of analytes of interest. In some embodiments, the sensor further comprises a counter electrode and/or a reference electrode.

In some embodiments, the presently disclosed subject matter provides a method of sensing an analyte of interest, the method comprising: (a) applying a sample to a working electrode of a sensor comprising a working electrode coated with a coating comprising a redox polymer of the presently disclosed subject matter or a blend thereof, optionally wherein the sample comprises a sample suspected of comprising the analyte; and (b) measuring current to provide an output signal indicative of the presence or absence of the analyte. In some embodiments, the sample is a biological sample, optionally a blood sample or interstitial fluid. In some embodiments, the analyte of interest is selected from glucose, a ketone, an alcohol, and a lactate.

In some embodiments, the analyte of interest is a ketone and the working electrode is coated with a coating comprising 3-hydroxybutyrate dehydrogenase. In some embodiments, the analyte of interest is glucose and the working electrode is coated with a coating comprising glucose dehydrogenase.

In some embodiments, the presently disclosed subject matter provides a method of preparing an electrode comprising a coating comprising a redox polymer of the presently disclosed subject matter or a blend thereof, the method comprising: (a) dissolving the redox polymer or blend in an aqueous solution to provide a polymer solution; (b) adding an enzyme to the polymer solution; (c) coating the polymer solution onto an electrode, to provide a coated electrode; and (d) drying the coated electrode. In some embodiments, the method further comprises adding a cross-linking agent to the polymer solution prior to step (c), optionally wherein the cross-linking agent is selected from the group comprising glutaraldehyde, glyoxal, ethyl carbodiimide hydrochloride, aziridine, polyethyleneimine, trimethylolpropanetriacrylate, and pentaerythritol glycidyl ether.

In some embodiments, the presently disclosed subject matter provides a compound having a structure of formula (I′):

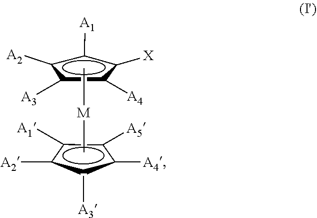

wherein: M is an atom of a metal element selected from the group comprising Fe, Ru, Mn, Os, V, Co, Sc, Ti, Cr, Cu, Zn, Ni, Mo, Rh, Pd, Cd, Pt, and Ir, optionally Fe; X is a substituent that comprises one or more reactive functional groups selected from the group comprising vinyl, alkenyl, OH, amino, epoxy, carboxylic acid, and isocyanate; A₁, A₂, A₃, and A₄ are each independently H or E, subject to the proviso that at least one of A₁, A₂, A₃, and A₄ is E; A₁′, A₂′, A₃′, A₄′, and A₅′ are each independently H or E, wherein at least two of A₁′, A₂′, A₃′, A₄′, and A₅′ are E; and E is an electron-donating aryl group substituent. In some embodiments, each E is independently selected from the group comprising alkyl, alkenyl, alkynyl, aralkyl, aryl, —N(R)₂, —NHR, —NH₂, —OH, —OR, —NHC(═O)R, and O—C(═O)R, wherein each R is independently selected from alkyl, aralkyl, and aryl.

In some embodiments, at least three of A₁, A₂, A₃, and A₄ are E and at least four of A₁′, A₂′, A₃′, A₄′, and A₅′ are E. In some embodiments, each of A₁, A₂, A₃, A₄, A₁′, A₂′, A₃′, A₄′, and A₅′ is E. In some embodiments, each E is C1-C10 alkyl. In some embodiments, each E is methyl.

It is an object of the presently disclosed subject matter to provide redox polymers, related electrodes, sensors, and monomers, as well as to provide related methods of detecting analytes of interest.

Certain objects of the presently disclosed subject matter having been stated hereinabove, which are addressed in whole or in part by the presently disclosed subject matter, other objects and aspects will become evident as the description proceeds when taken in connection with the accompanying Examples as best described herein below.

### BRIEF DESCRIPTION OF THE DRAWINGS

The presently disclosed subject matter can be better understood by referring to the following figures. The components in the figures are not necessarily to scale, emphasis instead being placed upon illustrating the principles of the presently disclosed subject matter. The drawings are not intended to limit the scope of this presently disclosed subject matter, which is set forth with particularity in the claims as appended or as subsequently amended, but merely to clarify and exemplify the presently disclosed subject matter.

For a more complete understanding of the presently disclosed subject matter, reference is now made to the below drawings.

FIG. 1 is a graph showing cyclic voltammograms (current (in microampere (μA)) versus potential (in volts (V) versus a silver/silver chloride (Ag/AgCl) reference electrode)) demonstrating the sensing of 3-hydroxybutyrate at different concentrations (0 milligrams per deciliter (mg/dL, solid line; 50 mg/dL, dark dashed line; 100 mg/dL, dark dashed and dotted line; and 200 mg/dL, light dashed line) by a sensor comprising an exemplary redox polymer of the presently disclosed subject matter.

FIG. 2 is a graph showing differential pulse voltammetry (DPV) plots (current (in microampere (μA)) versus potential (in volts (V) versus a silver/silver chloride reference electrode)) demonstrating sensing of 3-hydroxybutyrate at different concentrations (0 milligrams per deciliter (mg/dL, solid line; 50 mg/dL, dark dashed line; 100 mg/dL, dark dashed and dotted line; and 200 mg/dL, light dashed line) by a sensor comprising an exemplary redox polymer of the presently disclosed subject matter.

FIG. 3 is a graph showing sensor response (current (in microampere (μA) versus 3-hydroxybutyrate concentration (in milligrams per deciliter (mg/dL) at a potential of 0.06 volts (V) from the differential pulse voltammetry plots in FIG. 2.

FIG. 4 is a graph showing differential pulse voltammetry (DPV) plots (current (in microampere (μA)) versus potential (in volts (V) versus a silver/silver chloride reference electrode)) demonstrating sensing of glucose at different concentrations (0 milligrams per deciliter (mg/dL, solid line; 50 mg/dL, dark dashed line; 100 mg/dL, dark dashed and dotted line; 200 mg/dL, light dashed line; and 400 mg/dL, light dotted line) by a sensor comprising an exemplary redox polymer of the presently disclosed subject matter.

FIG. 5 is a graph showing sensor response (current (in microampere (μA) versus glucose concentration (in milligrams per deciliter (mg/dL) at a potential of 0.06 volts (V) from the differential pulse voltammetry plots in FIG. 4.

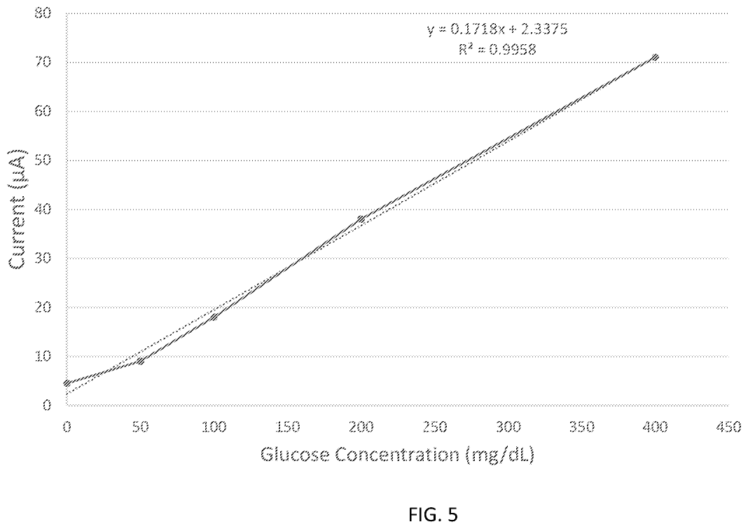

### DETAILED DESCRIPTION

The presently disclosed subject matter will now be described more fully hereinafter with reference to the accompanying Examples, in which representative embodiments are shown. The presently disclosed subject matter can, however, be embodied in different forms and should not be construed as limited to the embodiments set forth herein. Rather, these embodiments are provided so that this disclosure will be thorough and complete, and will fully convey the scope of the embodiments to those skilled in the art.

Unless otherwise defined, all technical and scientific terms used herein have the same meaning as commonly understood by one of ordinary skill in the art to which this presently described subject matter belongs. All publications, patent applications, patents, and other references mentioned herein are incorporated by reference in their entirety.

Throughout the specification and claims, a given chemical formula or name shall encompass all optical and stereoisomers, as well as racemic mixtures where such isomers and mixtures exist, unless as otherwise specifically indicated.

### I. Definitions

Following long-standing patent law convention, the terms “a”, “an”, and “the” refer to “one or more” when used in this application, including the claims. Thus, for example, reference to “a solvent” includes mixtures of one or more solvents, two or more solvents, and the like.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the present specification and attached claims are approximations that can vary depending upon the desired properties sought to be obtained by the presently disclosed subject matter.

The term “about”, as used herein when referring to a measurable value such as an amount of weight, molar equivalents, time, temperature, etc. is meant to encompass in one example variations of ±20% or +10%, in another example ±5%, in another example ±1%, and in yet another example ±0.1% from the specified amount, as such variations are appropriate to perform the disclosed methods.

The term “and/or” when used to describe two or more activities, conditions, or outcomes refers to situations wherein both of the listed conditions are included or wherein only one of the two listed conditions are included.

The term “comprising”, which is synonymous with “including,” “containing,” or “characterized by” is inclusive or open-ended and does not exclude additional, unrecited elements or method steps. “Comprising” is a term of art used in claim language, which means that the named elements are essential, but other elements can be added and still form a construct within the scope of the claim.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the phrase “consisting essentially of” limits the scope of a claim to the specified materials or steps, plus those that do not materially affect the basic and novel characteristic(s) of the claimed subject matter.

With respect to the terms “comprising”, “consisting of”, and “consisting essentially of”, where one of these three terms is used herein, the presently disclosed and claimed subject matter can include the use of either of the other two terms.

As used herein, a “macromolecule” refers to a molecule of high relative molecular mass (e.g., greater than 750 daltons, greater than 1000 daltons, greater than 5000 daltons, etc.), the structure of which comprises the multiple repetition of units derived from molecules of low relative molecular mass, e.g., monomers and/or oligomers.

An “oligomer” refers to a molecule of intermediate relative molecular mass, the structure of which comprises a small plurality of units derived from molecules of lower relative molecular mass.

As used herein, a “monomer” refers to a molecule that can undergo polymerization, thereby contributing constitutional units, i.e., a repeating group of atoms, to the structure of a macromolecule.

A “polymer” refers to a substance composed of macromolecules.

A “copolymer” refers to a polymer derived from more than one species of monomer. Copolymers include terpolymers (polymers prepared from three different monomers) and tetrapolymers (copolymers prepared from four different monomers).

As used herein, a “block macromolecule” refers to a macromolecule that comprises blocks in a linear sequence. A “block” refers to a portion of a macromolecule that has at least one feature that is not present in the adjacent portions of the macromolecule. A “block copolymer” refers to a copolymer in which adjacent blocks are constitutionally different, i.e., each of these blocks comprises constitutional units derived from different characteristic species of monomer or with different composition or sequence distribution of constitutional units.

For example, a diblock copolymer of polybutadiene and polystyrene is referred to as polybutadiene-block-polystyrene. Such a copolymer is referred to generically as an “AB block copolymer.” Likewise, a triblock copolymer can be represented as “ABA.” Other types of block polymers exist, such as multiblock copolymers of the (AB)ₙ type, ABC block polymers comprising three different blocks, and star block polymers, which have a central point with three or more arms, each of which is in the form of a block copolymer, usually of the AB type.

As used herein, a “graft macromolecule” refers to a macromolecule comprising one or more species of block connected to the main chain as side chains, wherein the side chains comprise constitutional or configurational features that differ from those in the main chain.

A “branch point” refers to a point on a chain at which a branch is attached. A “branch,” also referred to as a “side chain” or “pendant chain,” is an oligomeric or polymeric offshoot from a macromolecule chain. An oligomeric branch can be termed a “short chain branch,” whereas a polymeric branch can be termed a “long chain branch.”

A “chain” refers to the whole or part of a macromolecule, an oligomer, or a block comprising a linear or branched sequence of constitutional units between two boundary constitutional units, wherein the two boundary constitutional units can comprise an end group, a branch point, or combinations thereof.

A “linear chain” refers to a chain with no branch points intermediate between the boundary units.

A “branched chain” refers to a chain with at least one branch point intermediate between the boundary units.

A “main chain” or “backbone” refers to a linear chain from which all other chains are regarded as being pendant.

A “long chain” refers to a chain of high relative molecular mass.

A “short chain” refers to a chain of low relative molecular mass.

An “end group” refers to a constitutional unit that comprises the extremity of a macromolecule or oligomer and, by definition, is attached to only one constitutional unit of a macromolecule or oligomer.

A “comb macromolecule” refers to a macromolecule comprising a main chain with multiple trifunctional branch points from each of which a linear side chain emanates.

A “random copolymer” refers to a copolymer in which the sequential distribution of monomeric units from different monomers is random.

A “star polymer” refers to a polymer comprising a macromolecule comprising a single branch point from which a plurality of linear chains (or arms) emanate. A star polymer or macromolecule with “n” linear chains emanating from the branch point is referred to as an “n-star polymer.” If the linear chains of a star polymer are identical with respect to constitution and degree of polymerization, the macromolecule is referred to as a “regular star macromolecule.” If different arms of a star polymer comprise different monomeric units, the macromolecule is referred to as a “variegated star polymer.”

As used herein the term “alkyl” refers to aliphatic hydrocarbon groups, e.g., C1-C20 inclusive, linear (i.e., “straight-chain”), branched, or cyclic, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C1-C8 alkyl), e.g., 1, 2, 3, 4, 5, 6, 7, or 8 carbon atoms. In some embodiments, “lower alkyl” can refer to C1-C6 or C1-C5 alkyl groups. “Higher alkyl” refers to an alkyl group having about 10 to about 20 carbon atoms, e.g., 10, 11, 12, 13, 14, 15, 16, 17, 18, 19, or 20 carbon atoms. In certain embodiments, “alkyl” refers, in particular, to C1-C10 straight-chain or branched-chain saturated alkyl groups.

Alkyl groups can optionally be substituted (a “substituted alkyl”) with one or more alkyl group substituents, which can be the same or different. The term “alkyl group substituent” includes but is not limited to alkyl, substituted alkyl, halo, nitro, cyano, amino, arylamino, acyl, hydroxyl, aryloxyl, alkoxyl, alkylthio, arylthio, aralkyloxyl, aralkylthio, carboxyl, alkoxycarbonyl, oxo, and cycloalkyl. There can be optionally inserted along the alkyl chain one or more oxygen, sulfur or substituted or unsubstituted nitrogen atoms, wherein the nitrogen substituent is hydrogen, lower alkyl (also referred to herein as “alkylaminoalkyl”), or aryl.

Thus, as used herein, the term “substituted alkyl” includes alkyl groups, as defined herein, in which one or more atoms or functional groups of the alkyl group are replaced with another atom or functional group, including for example, alkyl, substituted alkyl, halogen, aryl, substituted aryl, alkoxyl, hydroxyl, nitro, cyano, amino, alkylamino, dialkylamino, ester, acyl, amide, sulfonyl, sulfate, and mercapto.

The term “alkenyl” refers to an alkyl group as defined above including at least one carbon-carbon double bond. Exemplary alkenyl groups include, but are not limited to, ethenyl, propenyl, butenyl, pentenyl, hexenyl, octenyl, butadienyl, and allenyl groups. The term “alkenyl” includes both alkenyl and cycloalkenyl groups. In some embodiments, alkenyl refers to a C1-C6 alkenyl group. Alkenyl groups can optionally be substituted with one or more alkyl group substitutents, which can be the same or different, including, but not limited to alkyl (saturated or unsaturated), substituted alkyl (e.g., halo-substituted and perhalo-substituted alkyl, such as but not limited to, —CF₃), cycloalkyl, halo, nitro, hydroxyl, carbonyl, carboxyl, acyl, alkoxyl, aryloxyl, aralkoxyl, thioalkyl, thioaryl, thioaralkyl, amino (e.g., aminoalkyl, aminodialkyl, aminoaryl, etc.), sulfonyl, and sulfinyl.

The term “vinyl” as used herein refers to a —CH═CH₂ group.

The term “alkynyl” refers to an alkyl group as defined above including at least one carbon-carbon triple bond. Examples of alkynyl groups include, but are not limited to, ethynyl, 1-propynyl, isopropynyl. The term “alkynyl” includes both alkynyl and cycloalkynyl groups.

“Cyclic” and “cycloalkyl” refer to a non-aromatic mono- or multicyclic ring system of about 3 to about 10 carbon atoms, e.g., 3, 4, 5, 6, 7, 8, 9, or 10 carbon atoms. In some embodiments, the cycloalkyl ring system comprises between 3 and 6 carbon atoms. The cycloalkyl group can be optionally partially unsaturated. The cycloalkyl group also can be optionally substituted with an alkyl group substituent as defined herein. There can be optionally inserted along the cyclic alkyl chain one or more oxygen, sulfur or substituted or unsubstituted nitrogen atoms, wherein the nitrogen substituent is hydrogen, alkyl, substituted alkyl, aryl, or substituted aryl, thus providing a heterocyclic group. Representative monocyclic cycloalkyl rings include, but are not limited to, cyclopropyl, cyclobutyl, cyclopentyl, cyclohexyl, cycloheptyl, and the like. Further, the cycloalkyl group can be optionally substituted with a linking group, such as an alkylene group as defined hereinbelow, for example, methylene, ethylene, propylene, and the like. In such cases, the cycloalkyl group can be referred to as, for example, cyclopropylmethyl, cyclobutylmethyl, and the like. Additionally, multicyclic cycloalkyl rings include adamantyl, octahydronaphthyl, decalin, camphor, camphane, and noradamantyl.

Thus, as used herein, the term “substituted cycloalkyl” includes cycloalkyl groups, as defined herein, in which one or more atoms or functional groups of the cycloalkyl group are replaced with another atom or functional group, including for example, alkyl, substituted alkyl, halogen, aryl, substituted aryl, alkoxyl, hydroxyl, nitro, cyano, amino, alkylamino, dialkylamino, ester, acyl, amide, sulfonyl, sulfate, and mercapto.

The term “arene” as used herein refers to a monocyclic or polycyclic aromatic hydrocarbon compound or group, i.e., a compound or group comprising one or more aromatic rings.

The term “aryl” is used herein to refer to an aromatic substituent that can be a single aromatic ring, or multiple aromatic rings that are fused together, linked covalently, or linked to a common group, such as, but not limited to, a methylene or ethylene moiety. The common linking group also can be a carbonyl, as in benzophenone, or oxygen, as in diphenylether, or nitrogen, as in diphenylamine. The term “aryl” specifically encompasses heterocyclic aromatic compounds (i.e., “heteroaryl”). The aromatic ring(s) can comprise phenyl, naphthyl, biphenyl, diphenylether, diphenylamine and benzophenone, among others. In particular embodiments, the term “aryl” means a cyclic aromatic comprising about 5 to about 10 carbon atoms, e.g., 5, 6, 7, 8, 9, or 10 carbon atoms, and including 5- and 6-membered hydrocarbon and heterocyclic aromatic rings.

The aryl group can be optionally substituted (a “substituted aryl”) with one or more aryl group substituents, which can be the same or different, wherein “aryl group substituent” includes alkyl, substituted alkyl, aryl, substituted aryl, aralkyl, hydroxyl, alkoxyl, aryloxyl, aralkyloxyl, carboxyl, acyl, halo, nitro, alkoxycarbonyl, aryloxycarbonyl, aralkoxycarbonyl, acyloxyl, acylamino, aroylamino, carbamoyl, alkylcarbamoyl, dialkylcarbamoyl, arylthio, alkylthio, alkylene, and —NR′R″, wherein R′ and R″ can each be independently hydrogen, alkyl, substituted alkyl, aryl, substituted aryl, and aralkyl.

Thus, as used herein, the term “substituted aryl” includes aryl groups, as defined herein, in which one or more atoms or functional groups of the aryl group are replaced with another atom or functional group, including for example, alkyl, substituted alkyl, halogen, aryl, substituted aryl, alkoxyl, hydroxyl, nitro, amino, alkylamino, dialkylamino, sulfate, and mercapto.

Specific examples of aryl groups include, but are not limited to, cyclopentadienyl, phenyl, furan, thiophene, pyrrole, pyridine, imidazole, benzimidazole, isothiazole, isoxazole, pyrazole, pyrazine, triazine, thiazole, pyrimidine, quinoline, isoquinoline, indole, carbazole, napthyl, and the like.

“Aralkyl” refers to an aryl-alkyl- or an-alkyl-aryl group wherein aryl and alkyl are as previously described and can include substituted aryl and substituted alkyl. Thus, “substituted aralkyl” can refer to an aralkyl group comprising one or more alkyl or aryl group substituents. Exemplary aralkyl groups include benzyl, phenylethyl, and naphthylmethyl.

“Alkylene” can refer to a straight or branched bivalent aliphatic hydrocarbon group having from 1 to about 20 carbon atoms, e.g., 1, 2, 3, 4, 5, 6, 7, 8, 9, 10, 11, 12, 13, 14, 15, 16, 17, 18, 19, or 20 carbon atoms. The alkylene group can be straight, branched or cyclic. The alkylene group also can be optionally unsaturated (i.e., include alkene or alkyne groups) and/or substituted with one or more “alkyl group substituents.” There can be optionally inserted along the alkylene group one or more oxygen, sulfur or substituted or unsubstituted nitrogen atoms (also referred to herein as “alkylaminoalkyl”), wherein the nitrogen substituent is alkyl as previously described. Exemplary alkylene groups include methylene (—CH₂—); ethylene (—CH₂—CH₂—); propylene (—(CH₂)₃—); cyclohexylene (—C₆H₁₀—); —CH═CH—CH═CH—; —CH═CH—CH₂—; —(CH₂)_{q}—N(R)—(CH₂)ᵣ—, wherein each of q and r is independently an integer from 0 to about 20, e.g., 0, 1, 2, 3, 4, 5, 6, 7, 8, 9, 10, 11, 12, 13, 14, 15, 16, 17, 18, 19, or 20, and R is hydrogen or lower alkyl; methylenedioxyl (—O—CH₂—O—); and ethylenedioxyl (—O—(CH₂)₂—O—). An alkylene group can have about 2 to about 3 carbon atoms and can further have 6-20 carbons.

“Arylene” refers to a bivalent aryl group, which can be substituted or unsubstituted.

The term “aralkylene” refers to a bivalent group that comprises a combination of alkylene and arylene groups (e.g., -arylene-alkylene-, alkylene-arylene-alkylene-, arylene-alkylene-arylene-, etc.).

Similarly, the terms “cycloalkylene”, “heterocycloalkylene” and “heteroarylene” refer to bivalent cycloalkyl, heterocyclic, and heteroaryl groups, which can optionally be substituted with one or more alkyl or aryl group substitutents.

As used herein, the term “acyl” refers to an organic carboxylic acid group wherein the —OH of the carboxylic acid group has been replaced with another substituent. Thus, an acyl group can be represented by RC(═O)—, wherein R is an alkyl, substituted alkyl, aralkyl, substituted aralkyl, aryl or substituted aryl group as defined herein. As such, the term “acyl” specifically includes arylacyl groups, such as a phenacyl group. Specific examples of acyl groups include acetyl (i.e., —C(═O)CH₃) and benzoyl.

“Alkoxyl” refers to an alkyl-O— group wherein alkyl is as previously described, including substituted alkyl. The term “alkoxyl” as used herein can refer to, for example, methoxyl, ethoxyl, propoxyl, isopropoxyl, butoxyl, t-butoxyl, and pentoxyl. The terms “oxyalkyl” and “alkoxy” can be used interchangably with “alkoxyl”.

“Aryloxyl” and “aryloxy” refer to an aryl-O— group wherein the aryl group is as previously described, including a substituted aryl. The term “aryloxyl” as used herein can refer to, for example, phenyloxy and naphthyloxy to alkyl, substituted alkyl, or alkoxyl substituted phenyloxy or naphthyloxy.

“Aralkyloxyl” or “aralkoxy” refer to an aralkyl-O— group wherein the aralkyl group is as previously described. An exemplary aralkyloxyl group is benzyloxyl.

The term “carbonyl” refers to the group —C(═O)—. The term “carbonyl carbon” refers to a carbon atom of a carbonyl group. Other groups such as, but not limited to, acyl groups, anhydrides, aldehydes, esters, lactones, amides, ketones, carbonates, and carboxylic acids, include a carbonyl group.

The terms “carboxyl” and “carboxylic acid” refer to the —C(═O)OH or —C(═O)O⁻ group.

The terms “halo” or “halogen” as used herein refer to fluoro (F), chloro (Cl), bromo (Br), and iodo (I) groups.

The term “haloalkyl” refers to an alkyl group as defined herein substituted by one or more halo groups.

The term “amide” refers to a compound comprising the structure R′—NR″—C(═O)—R, wherein R is alkyl, substituted alkyl, aralkyl, substituted aralkyl, aryl or substituted aryl, and wherein R′ and R″ are independently hydrogen, alkyl, aralkyl, or aryl, wherein the alkyl, aralkyl, or aryl are optionally substituted. In some embodiments, R′ is alkyl, substituted alkyl, aralkyl, substituted aralkyl, aryl, or substituted aryl.

The term “urea” as used herein refers to a compound comprising the structure R—NR′—C(═O)—NR′—R, wherein each R is independently alkyl, substituted alkyl, aralkyl, substituted aralkyl, aryl, or substituted aryl, and wherein each R′ is independently H, alkyl, substituted alkyl, aralkyl, substituted aralkyl, aryl, or substituted aryl.

A structure represented generally by a formula such as:

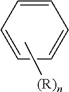

as used herein refers to a ring structure, for example, but not limited to a 3-carbon, a 4-carbon, a 5-carbon, a 6-carbon, and the like, aliphatic and/or aromatic cyclic compound comprising a substituent R group, wherein the R group can be present or absent, and when present, one or more R groups can each be substituted on one or more available carbon atoms of the ring structure. The presence or absence of the R group and number of R groups is determined by the value of the integer n. Each R group, if more than one, is substituted on an available carbon of the ring structure rather than on another R group. For example, the structure:

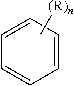

wherein n is an integer from 0 to 2 comprises compound groups including, but not limited to:

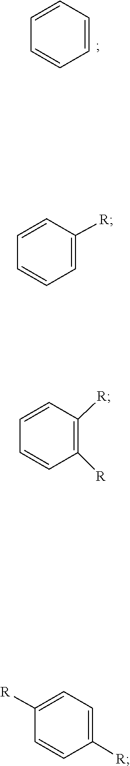

and the like.

When a named atom of an aromatic ring or a heterocyclic aromatic ring is defined as being “absent,” the named atom is replaced by a direct bond. When the linking group or spacer group is defined as being absent, the linking group or spacer group is replaced by a direct bond.

A line crossed by a wavy line, e.g., in the structure:

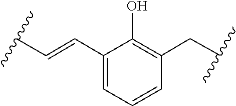 [SMILES: */C=C/c1cccc(C*)c1O]

indicates the site where a chemical moiety can bond to another group.

The term “amine” refers to a molecule having the formula N(R)₃, or a protonated form thereof, wherein each R is independently H, alkyl, substituted alkyl, aryl, substituted aryl, aralkyl, substituted aralkyl, or wherein two R groups together form an alkylene or arylene group. The term “primary amine” refers to an amine wherein at least two R groups are H. The term “secondary amine” refers to an amine wherein only one R group is H. The term “alkylamine” can refer to an amine wherein two R groups are H and the other R group is alkyl or substituted alkyl. “Dialkylamine” can refer to an amine where two R groups are alkyl. “Arylamine” can refer to an amine wherein one R group is aryl. Amines can also be protonated, i.e., have the formula

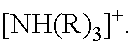

The term “amino” refers to the group —N(R)₂ wherein each R is independently H, alkyl, substituted alkyl, aryl, substituted aryl, aralkyl, or substituted aralkyl. The terms “aminoalkyl” and “alkylamino” can refer to the group —N(R)₂ wherein each R is H, alkyl or substituted alkyl, and wherein at least one R is alkyl or substituted alkyl. The term “dialkylamino” refers to an aminoalkyl group where both R groups are alkyl or substituted alkyl, which can be the same or different.

The terms “acylamino” and “aminoacyl” refer to the —N(R)—C(═O)R′ group, wherein R is selected from H, alkyl, substituted alkyl, aralkyl, substituted aralkyl, aryl, and substituted aryl, and wherein R′ is selected from alkyl, substituted alkyl, aralkyl, substituted aralkyl, aryl, and substituted aryl.

The terms “acyloxy” and “acyloxyl” refer to the —OC(═O)R group, wherein R is selected from H, alkyl, substituted alkyl, aralkyl, substituted aralkyl, aryl, and substituted aryl.

The term “cyano” refers to the —C≡N group.

The term “isocyanato” refers to the —N═C═O group.

The terms “hydroxyl” and “hydroxy” refer to the —OH group.

The term “oxo” refers to a compound described previously herein wherein a carbon atom is replaced by an oxygen atom.

The term “epoxy” refers to a group comprising a three-membered ring structure where the ring comprises two carbon atoms and one oxygen atom.

When the term “independently selected” is used, the substituents being referred to (e.g., R groups, such as groups R₁ and R₂, or groups X and Y), can be identical or different. For example, both R₁ and R₂ can be substituted alkyls, or R₁ can be hydrogen and R₂ can be a substituted alkyl, and the like.

A named “R”, “R′,” “X,” “Y,” “Y′”, “A,” “A′”, “B,” “L,” or “Z” group will generally have the structure that is recognized in the art as corresponding to a group having that name, unless specified otherwise herein. For the purposes of illustration, certain representative “R,” “X,” and “Y” groups as set forth above are defined below. These definitions are intended to supplement and illustrate, not preclude, the definitions that would be apparent to one of ordinary skill in the art upon review of the present disclosure.

The term “redox mediator” refers to an electron transfer agent, which carries electrons between an analyte (reduced or oxidized) and an electrode surface.

The term “organometallic redox mediator” as used herein can refer to an organometallic compound that act as a redox mediator. In some embodiments, the organometallic redox mediator is incorporated into (e.g., covalently attached to or non-covalently associated with) a polymer.

The term “organometallic compound” as used herein can refer to a compound that contains at least one bond between a metal center (e.g., e.g., a transition metal cation) and a carbon atom of an organic compound. The organic compound can be an arene, such as a single or multi-ring aromatic compound. In some embodiments, the organic compound is selected from cyclopentadienyl, cyclohexatrienyl, cycloheptatrienyl, cyclooctateraenyl, indenyl, tetrahydroindenyl, and fluorenyl. In some embodiments, the metal center is an atom of a transition metal, e.g., selected from the group including, but not limited to, Fe, Ru, Mn, Os, V, Co, Sc, Ti, Cr, Cu, Zn, Ni, Mo, Rh, Pd, Cd, Pt, and Ir.

The term “biological sample” as used herein, refers to a sample comprising a biological fluid, for example blood, interstitial fluid, spinal fluid, saliva, urine, tears, sweat, or the like, or a sample derived therefrom, e.g., via extraction or dilution of a biological fluid or tissue. Thus, a biological sample can be a biological fluid present in or taken from an animal, such as a human or other mammal.

### II. Redox Polymers

As described hereinabove, electrochemical sensors for the detection of analytes can include electron transfer redox mediators. For example, in electrochemical sensors that include enzymes (e.g., oxidases or dehydrogenases) that react with analytes of interest, the redox mediators present on or near an electrode surface can shuttle electrons and assist in electrical communication between the enzyme and the working electrode. In some embodiments, the redox mediator can improve the sensitivity and accuracy of the electrochemical sensor.

Among the various redox mediators that can be used in electrochemical sensors, the exemplary metallocene redox mediator ferrocene, which includes an iron (Fe) atom bonded to two cyclopentadienyl rings, has both fast electron transfer kinetics and is stable in both the oxidized and reduced forms. The reduction potential of ferrocene is 0.2 V (vs Ag/AgCl reference electrode). In this potential range, common interferents in biological samples, such as, acetaminophen, uric acid, ascorbic acid, and dopamine, among others, can permeate an electrode surface and interfere with the sensor signal for common target analytes of interest, such as glucose and ketones. At a lower reduction potential, (e.g., below 0.1 V), these interferents can be reduced or cannot permeate the electrode surface.

Accordingly, in some aspects, the presently disclosed subject matter provides an organometallic (e.g., a substituted ferrocene or other metallocene) redox mediator with a reduced reduction potential compared to the comparable unsubstituted organometallic redox mediator (e.g., unsubstituted ferrocene). These mediators can be used in electrochemical sensing applications with reduced interference from non-target analytes. In some embodiments, the organic group or groups of the organometallic redox mediator are substituted with electron-donating substituents, such as, but not limited to, alkyl (including saturated alkyl, alkenyl, and alkynyl groups), aralkyl, aryl, amino (e.g., —NH₂, alkylamino, or dialkylamino), hydroxy, alkoxy, aralkoxy, aryloxy, acylamino (e.g., —NHC(═O)R groups), and acyloxy (e.g., —OC(═O)R) groups. In some embodiments, the presently disclosed subject matter provides a polymerizable organometallic (e.g., ferrocene) compound that comprises one or more substituted organic groups bound to a metal center where the one or more substituted organic groups are together substituted by a plurality of electron-donating substituents (e.g., 2, 3, 4, 5, 6, 7, 8, 9, or 10 electron-donating substitents) and at least one substituent comprising one or more polymerizable functional groups (e.g., vinyl, alkenyl, halo, amino, hydroxy, epoxy, carboxylic acid, etc.). In some embodiments, the presently disclosed subject matter provides a polymer comprising one or more organometallic (e.g., ferrocene) redox mediators, each comprising one or more substituted organic groups bound to a metal center, where the one or more substituted organic groups are together substituted by a plurality of electron-donating substituents, and wherein the organometallic redox mediator or mediators is/are attached covalently or non-covalently (e.g., via ionic interactions, Van der Waals forces, and/or hydrogen bonding) to a polymeric chain in the polymer.

The polymer can have any structure. For example, the polymer can be a homopolymer, a copolymer (e.g., a random copolymer, a statistical copolymer, a block copolymer, a graft copolymer), a star polymer, a hyperbranched polymer, or a dendritic polymer. In some embodiments, the polymer is part of a polymer blend. The chemical class of the polymer is not particularly limited. The polymer can be hydrophilic, hydrophobic or amphiphilic. Suitable polymer classes include, but are not limited to, vinyl polymers (i.e., polymers prepared from vinyl group-containing monomers), polyolefins, polyethers, polyamine polymers, polyesters, polyamides, polycarbonates, polyureas, polyurethanes, polysaccharides, epoxys, etc.

Thus, in some embodiments, the presently disclosed subject matter provides a redox polymer, i.e., a polymeric material that comprises a redox mediator, typically a plurality of the same redox mediator or a combination of one or more of each of a plurality of different redox mediators. In some embodiments, each redox mediator comprises a metallocene (e.g., ferrocene). In some embodiments, the redox polymer comprises: at least one polymeric chain; and a plurality of metallocene groups, wherein each of the plurality of metallocene groups comprises a metal center (e.g., a metal atom) bound to two arene groups, wherein the two arene groups are together substituted by at least three electron-donating substituents and one substituent that comprises a linker group, wherein the linker group is covalently or non-covalently bound to the at least one polymeric chain. In some embodiments, the two arene groups are together substituted by at least four electon donating substituents, wherein one of the electron-donating substituents is itself substituted by one or more polymerizable functional groups or at least one group that can non-covalently interact with a polymer chain. Stated another way, in some embodiments, the substituent comprising the linker group is also an electron-donating substituent. Thus, in some embodiments, the two arene groups of each of the plurality of metallocene groups are together substituted by at least four electron-donating substitutents, one of which is reactive in a polymerization reaction.

In some embodiments, the two arene groups can be independently selected from cyclopentadienyl, cyclohexatrienyl, cycloheptatrienyl, cyclooctateraenyl, indenyl, tetrahydroindenyl, and fluorenyl. In some embodiments, the two arene groups can be bonded to one another via a bivalent linker group (e.g., alkylene group, such as methylene or dimethylmethylene). In some embodiments, each of the two arene groups are cyclopentadienyl (Cp) groups.

In some embodiments, the metal center is an atom of a transition metal, a post-transition metal, a lanthanide, or an actinide. In some embodiments, the metal center is a transition metal atom. In some embodiments, the metal center is an atom of a metal selected from the group comprising Fe, ruthenium (Ru), manganese (Mn), osmium (Os), vanadium (V), cobalt (Co), scandium (Sc), titanium (Ti), chromium (Cr), copper (Cu), zinc (Zn, nickel (Ni), molybdenum (Mo), rhodium (Rh), palladium (Pd), cadmium (Cd), platinum (Pt), and iridium (Ir). In some embodiments, the metal center is a Fe atom.

In some embodiments, one or more of the plurality of metallocene groups has a redox potential of less than about 0.2 volts (V) (versus Ag/AgCl reference electrode). Thus, for instance, in some embodiments, one or more of (e.g., each of) the plurality of metallocene groups has a redox potential of about 0.19 V or less, about 0.18 V or less, 0.17 V or less, 0.16 V or less, 0.15 V or less, 0.14 V or less, 0.13 V or less, 0.12 V or less, or 0.11 V or less versus an Ag/AgCl reference electrode. In some embodiments, one or more (e.g., each of) the plurality of metallocene groups has a redox potential of about 0.1 V or less versus an Ag/AgCl reference electrode.

In some embodiments, each of the plurality of metallocene groups has a structure of formula (I):

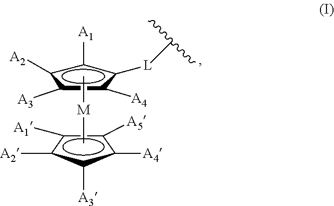

wherein: M is a metal atom of a element selected from the group comprising Fe, Ru, Mn, Os, V, Co, Sc, Ti, Cr, Cu, Zn, Ni, Mo, Rh, Pd, Cd, Pt, and Ir; L is a linking group that is covalently bound to the at least one polymeric chain; A₁, A₂, A₃, and A₄ are each independently H or E, subject to the proviso that at least one of A₁, A₂, A₃, and A₄ is E; A₁′, A₂′, A₃′, A₄′, and A₅′ are each independently H or E, wherein at least two of A₁′, A₂′, A₃′, A₄′, and A₅′ are E; and each E is an electron-donating substituent (which can be the same or different). Thus, the metallocene comprises at least four substituents between the two cycopentadienyl rings (i.e., is a tetra-substituted metallocene). In some embodiments, each E is independently selected from the group comprising alkyl (e.g., saturated alkyl, alkenyl, or alkynyl), aralkyl, aryl, —N(R)₂, —NHR, —NH₂, —OH, —OR, —NHC(═O)R, and —OC(═O)R, wherein each R is independently selected from substituted or unsubstituted alkyl, aralkyl, and aryl.

In some embodiments, at least three of A₁, A₂, A₃, and A₄ are E and at least four of A₁′, A₂′, A₃′, A₄′, and A₅′ are E. Thus, in some embodiments, the metallocene group of formula (I) is an octa-substituted metallocene. In some embodiments, at least two or at least three of A₁, A₂, A₃, and A₄ are alkyl (e.g., methyl) and at least three or at least four of A₁′, A₂′, A₃′, A₄′, and A₅′ are alkyl (e.g., methyl). In some embodiments, each of A₁, A₂, A₃, A₄, A₁′, A₂′, A₃′, A₄′, and A₅′ is E. Thus, in some embodiments, the metallocene group of formula (I) is deca-substituted. In some embodiments, each E is C1-C10 alkyl (e.g., methyl, ethyl, —CH═CHCH₃, —CH═CH(CH₂CH₃), etc). In some embodiments, each E is methyl.

The linking group L of formula (I) is not particularly limited, but can be any suitable bivalent group (e.g., substituted or unsubstituted alkylene, cycloalkylene or aralkylene). In some embodiments, L comprises a —CH═CH—(CH₂)ₙ— group, wherein n is an integer between 1 and 9 (e.g., 1, 2, 3, 4, 5, 6, 7, 8, or 9). In some embodiments, L is selected from the group comprising:

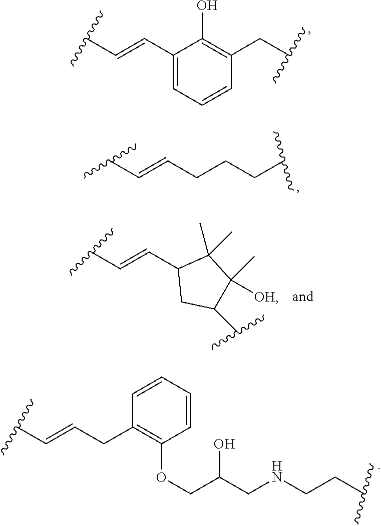

In some embodiments, the redox polymer is a homopolymer. In some embodiments, the redox polymer is a copolymer (e.g., a random copolymer, graft copolymer, or block copolymer) prepared by polymerization of (i) a monomer comprising a metallocene comprising cyclopentadienyl groups substituted by a plurality (e.g., at least 3 or at least 4) electron-donating groups, and (ii) one or more additional, non-metallocene-containing monomers. The polymerization method is not particularly limited and can involve, for example, radical polymerization, condensation polymerization, ionic polymerization, atom transfer radical polymerization (ATRP), or reversible addition-fragmentation chain transfer (RAFT) polymerization, depending upon the functional group or groups available for polymerization in the monomers.

Thus, in some embodiments, the presently disclosed redox polymers can be prepared from redox mediator monomers, i.e., polysubstituted metallocene compounds functionalized with a substituent comprising one or more polymerizable functional groups (e.g., vinyl, alkenyl, epoxide, amino, hydroxy, halo, carboxylic acid, etc.). Suitable polysubstituted metallocene compounds that can be used in the synthesis of the monomers can be prepared by methods known in the art. For example, polysubstituted ferrocenes can be prepared by deprotonating the corresponding polysubstituted cyclopentadiene with a base (e.g., potassium hydroxide or diethylamine) to form an anion, followed by reaction with an iron salt (e.g., FeCl₂) to form the polysubstituted ferrocene. Ferrocenes can also be prepared from Grignard reagents, i.e., by reaction of a cyclopentadienyl magnesium bromide and an iron salt. A variety of methods are also known in the art to modify ferrocene and other metallocenes by adding or transforming substituents on the cyclopentadienyl or other arene groups after the metallocene is synthesized. For example, the cyclopentadienyl moieties of ferrocene can undergo aromatic substitution reactions (e.g., Friedel-Craft alkylation or acylation) or other reactions of aromatic compounds. Additionally, metallocenes can be prepared via exchanging one metal center for another via transmetallation. For example, a ferrocene can be reacted with a manganese complex to provide a manganocene.

In some embodiments, the redox mediator monomer can be prepared by modifying one methyl substituent of a polymethylated metallocene, e.g., tetramethylferrocene, hexamethylferrocene, octamethylferrocene, or decamethylferrocene, to transform that methyl substituent into a substituent containing a polymerizable functional group, e.g., a vinyl, alkenyl, epoxy, carboxylic acid, amino, halo, or hydroxyl group. In some embodiments, the monomer can be prepared by modifying one methyl substituent of a commercially available polymethylated metallocene, such as, but not limited to, octamethylferrocene (Me₈Fc) and decamethylferrocene (Me₁₀Fc) to provide a redox mediator monomer.

For instance, Scheme 1, below, shows an exemplary synthetic approach to preparing a redox mediator monomer of the presently disclosed subject matter from an exemplary polysubstituted ferrocene, i.e., Me₁₀Fc. More particularly, as shown in Scheme 1, Me₁₀Fc can be contacted with 2,3-dichloro-5,6-dicyano-1,4-benzoquinone (DDQ) to provide aryl ether compound 1. Compound 1 can be transformed into phosphonate 2 via reaction with a trialkyl phosphite (e.g., triethylphosphite). Phosphonate 2 can then be contacted with an aldehyde, i.e., 3-allyl salicylaldehyde (3), in the presence of a base (e.g., an alkoxide, such as potassium tert-butoxide) to provide vinylic monomer 4 via a Horner-Wadsworth-Emmons (HWE) reaction. Monomer 4 can be polymerized via any suitable polymerization method for polymerizing compounds comprising carbon-carbon double bonds, e.g., free radical polymerization initiated by a radical initiator, such as ammonium persulfate (APS), an organic or inorganic peroxide, or an azo compound.

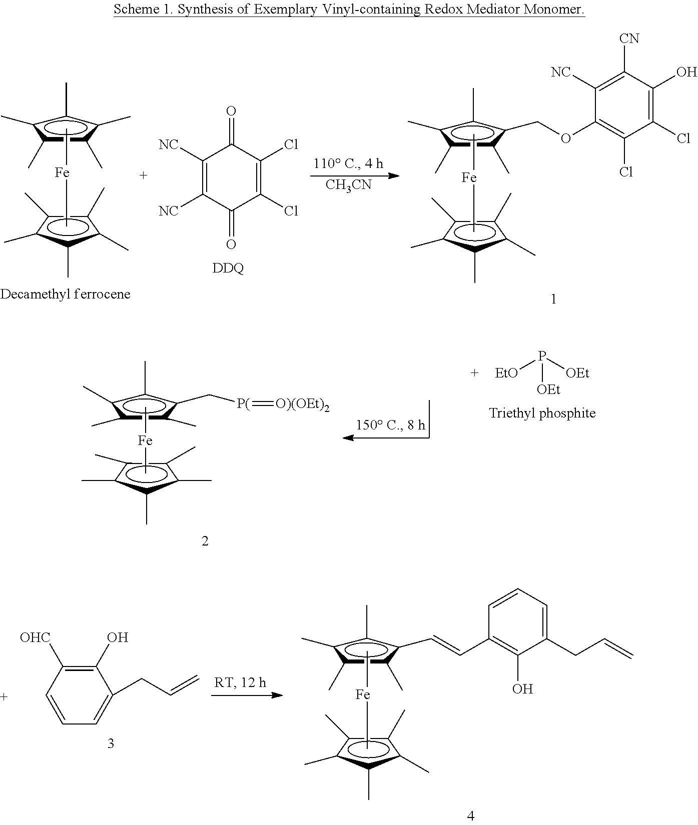

While the aldehyde in Scheme 1 is 3-allyl salicylaldehyde, which can provide a redox mediator monomer that when polymerized provides a polymer that includes an aralkylene linker, a variety of other alkene group-containing aldehydes can be used in place of aldehyde 3. For example, 3-allyl salicylaldehyde can be replaced with an unsaturated aliphatic aldehyde, such as 4-pentenal (5), as shown in Scheme 2, below, which can provide a metallocene monomer (6) that would provide a polymer where the linker L in the of formula (I) is alkylene. Other suitable aliphatic aldehydes include, but are not limited to, for example, 3-butenal and 5-hexenal.

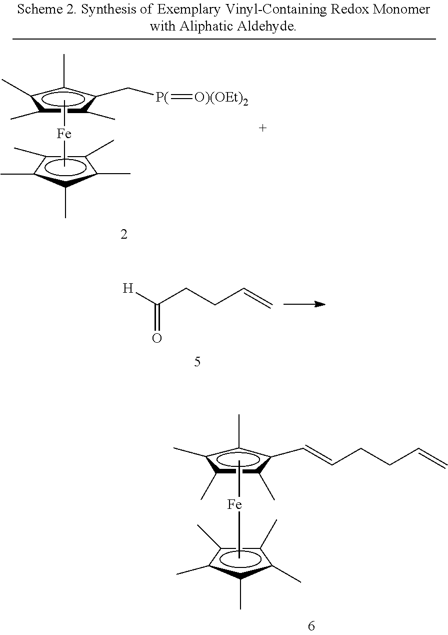

Moreover, the aldehydes that can be reacted with phosphonate 2 are not limited to those that contain alkene groups (e.g., vinyl or other terminal alkene groups). The aldehydes can also contain other functional groups (e.g., other terminal functional groups), such as, but not limited to, hydroxy, amino, acrylic, methacrylic, epoxy, carboxylic acid, and other polymerizable functional groups. In some embodiments, the aldehyde can include two amino groups, two hydroxy groups or both amino and hydroxy groups, e.g., to prepare a monomer that can be used in the preparation of urethane/urea polymers. For instance, Scheme 3, below, shows the preparation of a functionalized metallocene with a substitutent containing both an amine group and a hydroxy group. More particularly, in Scheme 3, aldehyde 3 from Scheme 1 is replaced by 4-amino-3-hydroxy-butanal (7), which provides monomer 8. Monomer 8 can be reacted with a co monomer diisocyanate (e.g., isophorane diisocyanate) to provide a diisocyanate prepolymer 9.

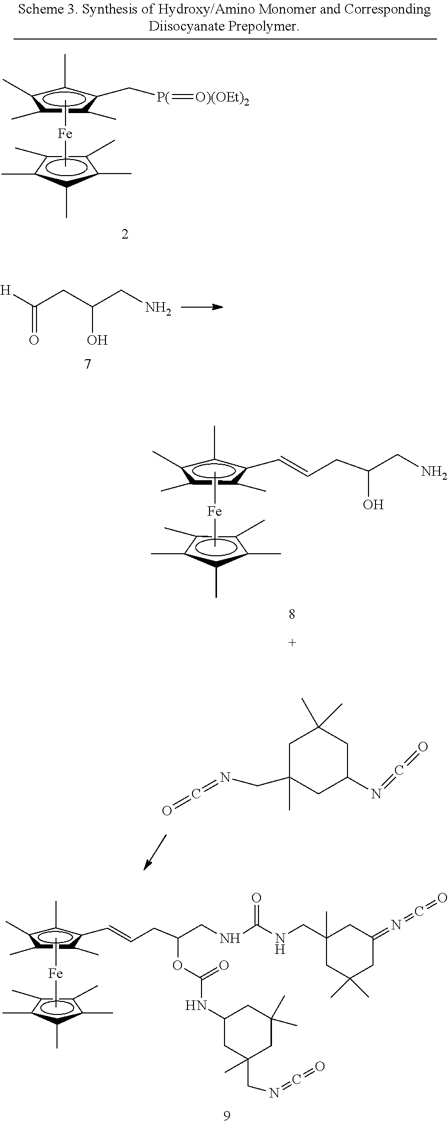

As an alternative to the cycloaliphatic diisocyanate shown in Scheme 3, other diisocyanates or mixtures of diisocyanates can be used. These can include, for example, other cyloaliphatic diisocyanates, aliphatic diisocyanates, aromatic diisocyanates, oligomeric diisocyanates, and polymeric diisocyanates. Further, the hydroxy and amino-functionalized metallocene monomer can be polymerized with the diisocyanate “as-is” or can be reacted with a chain extender, e.g., an aromatic or aliphatic, synthetic or bio-based diol or polyol, and then polymerized with diisocyanate. The diol or polyol chain extender can have any desired structure, e.g., linear, hyperbranched, dendrimer, or star.

Scheme 4, below, shows the preparation of an epoxide-containing monomer and its subsequent reaction with an amino group of a polyamine, such as poly(ethyleneimine) (PEI). As shown in Scheme 4, phosphonate 2 from Scheme 1 is reacted with an epoxide-containing aldehyde, i.e., epoxycamphorenic aldehyde (10), to provide epoxide-containing monomer 11, which can react with free amino groups in PEI.

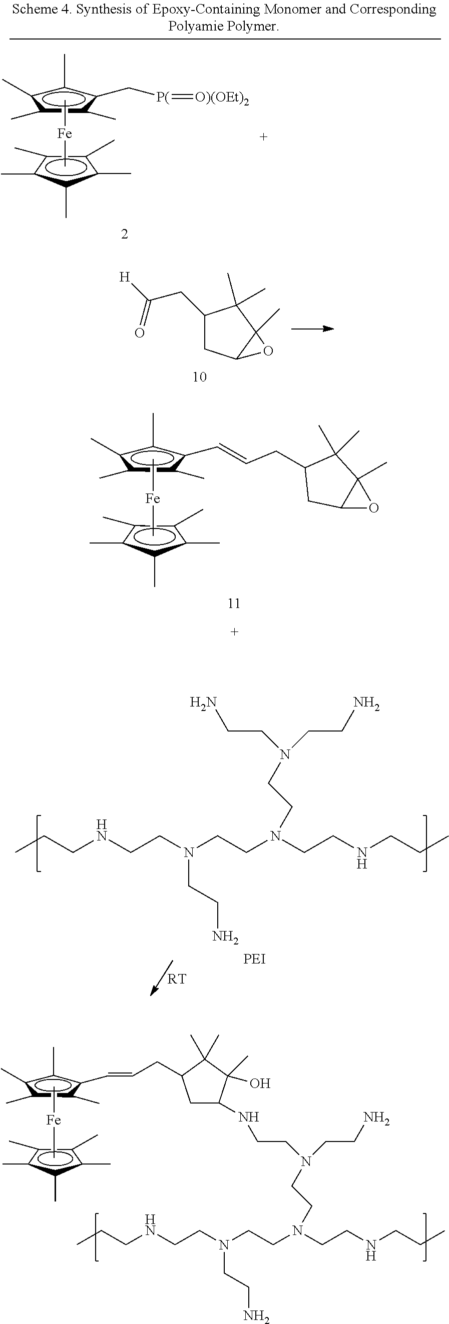

Further, additional redox mediator epoxy-containing or vinyl-containing monomers can be prepared as shown in Scheme 5, below, by synthetic elaboration of a hydroxy-containing aldehyde, such as 2-hydroxybenzaldehyde or another hydroxybenzaldehyde. For instance, as shown in Scheme 5, a hydroxy-containing aldehyde (e.g., 2-hydroxybenzaldehyde (12)) can be reacted with a halo-substituted epoxide compound (e.g., 2-(chloromethyl)oxirane (13)) to provide an epoxy-containing aldehyde (e.g., 2-(oxiran-2-ylmethoxy)benzaldehyde (14)). The epoxide can be opened with an amine, e.g., but-3-en-1-amine (15), to provide a vinyl-containing aldehyde (16) that can then undergo a HWE reaction with phosphonate 2 from Scheme 1, thereby providing a vinyl-containing redox mediator monomer (17). The hydroxy group in monomer 17 can be useful as a site for hydrogen bonding or crosslinking, e.g., in homopolymers prepared from monomer 17 or in compolymers without other hydrogen bonding or other crosslinkable groups. The amine 15 can be replaced by any suitable amine or amide compound that also contains a vinyl or acrylic group. For example, in addition to but-3-en-1-amine as shown in Scheme 5, the amine could be selected from an amine from the group including, but not limited to, acrylamide, allylamine, N-allylmethylamine, 2-methyl-2-propen-1-amine, N-(prop-2-en-1-yl)acetamide, 2-aminoethylmethacrylate, N-2-aminoethyl methacrylamide, N-(3-(N,N,-dimethylamino)propyl methacrylate, N-(2-N,N-dimethylamino)ethyl metacrylamide, N-(3-(N,N-dimethylamino)propyl acrylamide, and 2-(tert-butylamino)ethyl methacrylate. In addition, compound 14 or another epoxy-containing aldehyde can be reacted with phosphonate 2 to provide an epoxy-containing redox mediator monomer.

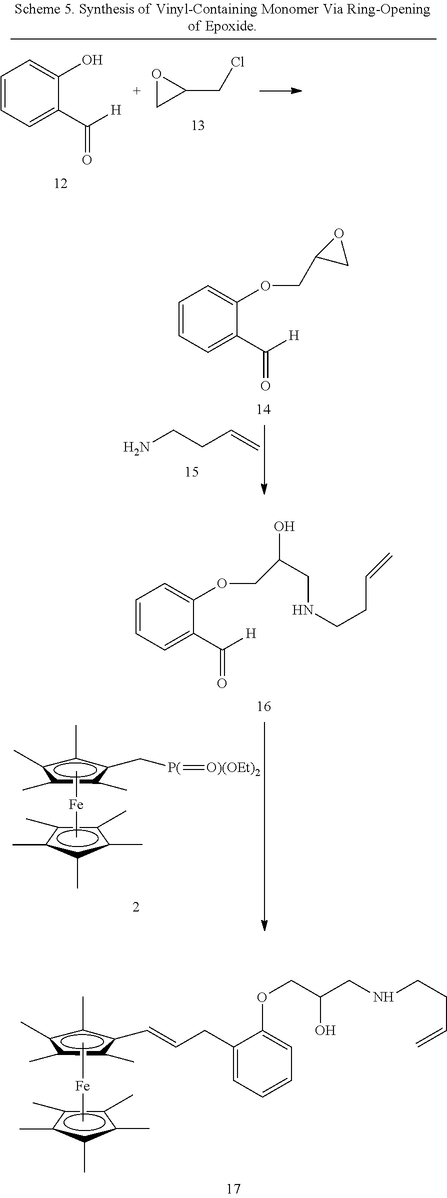

The redox mediator monomers can be used in the synthesis of homopolymers or copolymers. In some embodiments, the presently disclosed subject matter provides a redox homopolymer, where the redox homopolymer is a homopolymer prepared by polymerization of a metallocene-containing monomer comprising one or more reactive functional groups. In some embodiments, the homopolymer can be a polymerization product of a monomer have a structure of formula (I′):

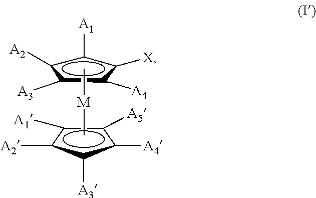

wherein: M is a metal atom (e.g., an atom of an element selected from the group comprising Fe, Ru, Mn, Os, V, Co, Sc, Ti, Cr, Cu, Zn, Ni, Mo, Rh, Pd, Cd, Pt, and Ir); X is a moiety that comprises one or more reactive functional groups; A₁, A₂, A₃, and A₄ are each independently H or E, subject to the proviso that at least one of A₁, A₂, A₃, and A₄ is E; A₁′, A₂′, A₃′, A₄′, and A₅′ are each independently H or E, wherein at least two of A₁′, A₂′, A₃′, A₄′, and A₅′ are E; and each E is an electron-donating aryl group substituent. In some embodiments, M is an Fe atom. In some embodiments, X comprises one or more functional group selected from vinyl, alkenyl, hydroxy, amino, epoxy, carboxylic acid, and —N═C═O. In some embodiments, X comprises -L′-X′, wherein L′ is a bivalent functional group (e.g., alkylene, cycloalkylene, or aralkylene) and X′ is —CH═CH₂, epoxy, or —CH(OH)—CH₂NH₂.

In some embodiments, the polymer has a structure of the formula (II):

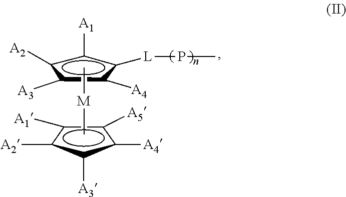

where M, A₁, A₂, A₃, A₄, A₁′, A₂′, A₃′, A₄′, A₅′ and L are as defined above for formula (I), n is an integer greater than 1 (e.g., at least 5, at least 10, at least 25, at least 50, at least 100, at least 250, at least 500 or more); and P is a bivalent group that forms part of the polymer backbone. For example, in some embodiments, the polymer is a vinyl polymer or a polyolefin and P is ethylene, i.e., —CH—CH—, or another alkylene. In some embodiments, the redox polymer has a structure of formula (II-1):

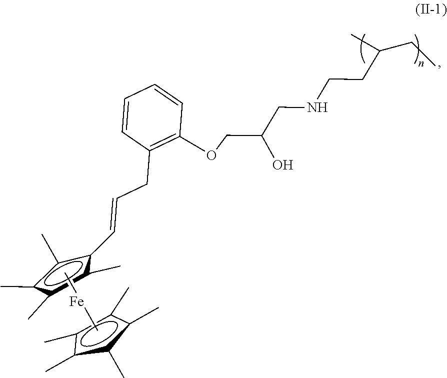

where n is an integer greater than 1. The polymer of formula (II-1) can be prepared, for example, by the polymerization of compound 17 in Scheme 5 above, using a radical initiator, such as ammonium persulfate (APS).

In some embodiments, the redox polymer is a copolymer prepared by copolymerization of at least two different monomers, wherein one of the monomers is a metallocene-containing monomer having a structure of formula (I′):

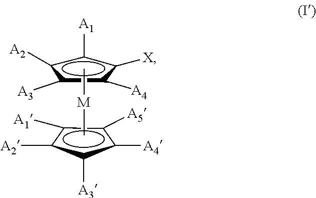

wherein: M is a metal atom (e.g., a metal atom of a metal selected from the group consisting of Fe, Ru, Mn, Os, V, Co, Sc, Ti, Cr, Cu, Zn, Ni, Mo, Rh, Pd, Cd, Pt, and Ir); X is a polymerizable moiety that comprises one or more reactive functional groups (e.g., vinyl, alkenyl, OH, amino, epoxy, —COOH, and —N═C═O); A₁, A₂, A₃, and A₄ are each independently H or E, subject to the proviso that at least one of A₁, A₂, A₃, and A₄ is E; A₁′, A₂′, A₃′, A₄′, and A₅′ are each independently H or E, wherein at least two of A₁′, A₂′, A₃′, A₄′, and A₅′ are E; and E is an electron-donating substituent. In some embodiments, M is a Fe atom. In some embodiments, the copolymer is a random copolymer, a block copolymer or a graft copolymer.

In some embodiments, the copolymer is prepared from a monomer mixture comprising about 1 wt % to about 50 wt % of the monomer of formula (I′) with respect to the total weight of the other monomer or monomers in the used to prepare the copolymer (e.g., about 1 wt %, about 5 wt %, about 10 wt %, about 15 wt %, about 20 wt %, about 25 wt %, about 30 wt %, about 35 wt %, about 40 wt %, about 45 wt %, or about 50 wt % of the monomer of Formula (I′) with respect to all monomers). In some embodiments, the copolymer is prepared from monomers that comprise about 1 wt % to about 20 wt % of a monomer of formula (I′) (e.g., about 1 wt %, about 2 wt %, about 3 wt %, about 4 wt %, about 5 wt %, about 6 wt %, about 7 wt %, about 8 wt %, about 9 wt %, about 10 wt %, about 11 wt %, about 12 wt %, about 13 wt %, about 14 wt %, about 15 wt %, about 16 wt %, about 17 wt %, about 18 wt %, about 19 wt %, or about 20 wt % of a monomer of formula (I′) with respect to all monomers).

In some embodiments, at least one of the one or more monomers (i.e., a monomer that is not a monomer of Formula (I′)) is water soluble and/or contains one or more hydrophilic groups (e.g., one or more ether linkages, a hydroxyl group, an amino group, a carboxyl group, etc.) or is a zwitterionic monomer (e.g., monomers comprising sulfobetaine groups, carboxybetaine groups, or phosphobetaine groups). In some embodiments, the copolymer is a terpolymer prepared by copolymerization of a metallocene-containing monomer (e.g., a metallocene-containing monomer having a structure of formula (I′) above), a zwitterionic monomer, and a third monomer. In some embodiments, the third monomer is a water-soluble monomer. In some embodiments, the three monomers each comprise a polymerizable carbon-carbon double bond, e.g., a vinyl, acrylic, or methacrylic group. In some embodiments, the terpolymer is prepared from a mixture of three monomers: (i) a zwitterionic monomer; (ii) a water-soluble monomer; and (iii) a monomer having a structure of formula (I′), wherein the mixture comprises the zwitterionic monomer and the water-soluble monomer in a weight ratio (zwitterionic monomer:water-soluble monomer) of about 1:9 to about 9:1 and further comprises about 1 wt % to about 50 wt % of the monomer of formula (I′) with respect to the combined weight of the other two monomers. In some embodiments, the weight ratio of zwitterionic monomer to water-soluble monomer is 3:1 and the monomer mixture comprises about 5 wt % of the monomer of formula (I′) with respect to the combined weight of the zwitterionic monomer and the water-soluble monomer.

In some embodiments, the monomer of formula (I′) is compound 4 from Scheme 1, above; the zwitterionic monomer is sulfobetaine methacrylate (SBMA), and the water-soluble monomer is 2-hydroxy ethylacrylate (HEA). Thus, in some embodiments, the copolymer has a structure of Formula (III):

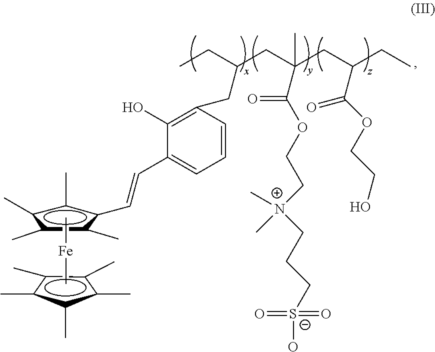

where x, y, and z are each independently integers greater than 1.

The presently disclosed polymers (e.g., copolymers) can be cross-linked or non-crosslinked. If crosslinked, suitable crosslinking agents include, but are not limited to, glutaraldehye, glyoxal, ethyl carbodiimide hydrochloride, aziridine, polyethyleneimine, trimethylolpropanetriacrylate and pentaerythritol glycidyl ether.

The presently disclosed polymers (e.g. homopolymers and copolymers) can be used alone or can be blended with or copolymerized with one or more additional polymers, such as, but not limited to, polyethylene glycol (PEG); polyvinylpyrrolidone (PVP); polyvinyl alcohol (PVA); poly(acrylic acid) (PAA), a polyacrylamide; N-(2-hydroxypropyl) methacrylamide (HPM); polyoxazoline; a polyphosphate; a polyphosphazene; a zwitterionic polymer; a polymer of a water-soluble methacrylate; a water-soluble natural polymer; a water-soluble acrylic monomer or a hydrophilic polymer, such as, but not limited to, poly(2-ethyl)oxazoline methacrylate, poly (2-ethyl-2-oxazoline)-poly(ethylene oxide)-poly(2-ethyl-2-oxazoline), poly(2-methyl-2-oxazoline), poly(2-methyl) oxazoline methacrylate, poly (2-nonyl-2-oxazoline), poly(2-oxazoline), poly (2-phenyl-2-oxazoline), poly(2-alkyl-2-oxazoline), poly(2-methyl-2-oxazoline)-co-poly(2-(3-butenyl)-2-oxazoline), a copolymer of polyoxazoline, a zwitterionic copolymer and/or a zwitterionic interpenetrating network (IPN) of poly(sulfobetaine methacrylate-2-oxazoline), poly(carboxybetaine methacrylate-2-oxazoline) and poly(phosphobetaine methacrylate-2-oxazoline), a hybrid of polysilsesquioxane and polyoxazoline; poly(2-acrylamido-2-methyl-1-propanesulfonic acid, poly(acrylamidomethylsulfonic acid), poly(methacrylamide-co-2-acrylamido-2-methyl-1-propanesulfonic acid), poly(methyl methacrylate-2-acrylamido-2-methylpropane sulfonic acid), poly(methyl ethacrylate-co-2-acrylamido-2-methylpropane sulfonic acid), poly(sulfobetaine-2-acrylamido-2-methylpropanesulfonic acid-co-acrylic acid), poly(N-isopropylacrylamide-2-(acrylamido)-2-methyl propane sulfonic acid, poly(itaconic acid-co-2-acrylamido-2-methylpropane sulfonic acid), poly(sulfobetaine methacrylate-2-acrylamido-2-methylpropane sulfonic acid), poly(carboxybetaine methacrylate-2-acrylamido-2-methylpropane sulfonic acid), poly(phosphobetaine methacrylate-2-acrylamido-2-methylpropane sulfonic acid), poly(methyl vinyl ether-co-maleic anhydride), IPN of poly(methyl vinyl ether-co-maleic anhydride) and polyoxazoline, IPN of poly(methyl vinyl ether-co-maleicanhydride) and poly(sulfobetaine methacrylate), IPN of poly(methyl vinyl ether-co-maleic anhydride) and poly(carboxybetaine methacrylate), IPN of poly(methyl vinyl ether-co-maleic anhydride) and poly(phosphobetaine methacrylate), poly(vinylphosphonic acid) and polyphosphoester; a homo- or copolymers of a polyamide (e.g., a polymer derived from amino acids), a polyester, a polyanhydride, a poly(ortho ester), a poly(amido amine), a poly(β-amino ester), collagen, cellulose, an alginate, a dextran, chitosan, polytetrafluoroethylene (PTFE), polyethylene, polypropylene, polymethylmethacrylate (PMMA), a polycarbonate, polyglycolic acid, polycaprolactone, a polymer of isoprene, suberin, lignin, cutin, cutan, melanin, a polynucleotide, a polypeptide, and a polysaccharide.

In some embodiments, the presently disclosed subject matter provides a blend comprising a redox polymer or copolymer as described herein and one or more additional polymers. In some embodiments, one or more of the one or more additional polymers are water soluble.

In some embodiments, the presently disclosed subject matter provides a solution comprising the redox polymer, an enzyme (e.g., that catalyzes a reaction of an analyte of interest, e.g., glucose or ketone), and optionally, a crosslinking agent. In some embodiments, the solution is an aqueous solution. In some embodiments, the solution comprises about 10 wt % to about 50 wt % of the redox polymer. The amount of polymer can be adjusted, for example, based upon the weight of the polymer and/or the desired viscosity of the solution. The solution can be used as a coating solution to coat a solid surface, e.g., a conductive sheet, wire or other object.

In some embodiments, the presently disclosed subject matter provides a composition comprising a redox polymer and an enzyme. In some embodiments, the composition comprising the redox polymer and the enzyme is a gel or a solid (e.g., is a solid membrane). In some embodiments, the redox polymer is crosslinked.

In some embodiments, the presently disclosed subject matter provides an electrode, wherein the electrode comprises a coating comprising a redox polymer as described hereinabove (e.g., a redox homopolymer, copolymer or a blend thereof). The electrode can be prepared from any suitable conductive material, such as, but not limited to carbon, palladium (Pd), platinum (Pt), an oxide of platinum, graphene oxide, graphite, graphene, a conductive polymer, a reduced graphene oxide. In some embodiments, the electrode is a metal or conductive polymer wire. The electrode thus coated can act as a working electrode to interact with an analyte of interest, such as a blood analyte (e.g., glucose, ketone, alcohol, lactate or the like). The coating can interact with the analyte or a product of an enzyme reaction with the analyte, for instance, resulting in reduction of the redox mediator of the redox polymer. Oxidation of the redox mediator at the surface of the electrode can then produce a measurable electric current that can be used to determine the amount of analyte present.

The technique of coating the electrode with the redox polymer is not particularly limited. In some embodiments, the electrode can be coated using dip coating, spin coating, or spray coating, for example. In some embodiments, the coating has a thickness of about 1 μmicrometer (μm) to about 20 μm (e.g, about 1, 2, 3, 4, 5, 6, 7, 8, 9, 10, 11, 12, 13, 14, 15, 16, 17, 18, 19, or about 20 μm). In some embodiments, the coating has a thickness of about 2 μm to about 5 μm.

In some embodiments, the coating further comprises an enzyme. The enzyme can be any enzyme that reacts with an analyte of interest (e.g., alcohol, lactate, ketone, glucose, etc.). In some embodiments, the enzyme is an oxidase or a dehydrogenase. In some embodiments, the enzyme is an enzyme that reacts with glucose or a ketone body (e.g., 3-hydroxybutyrate). In some embodiments, the enzyme is selected from the group comprising glucose oxidase, glucose dehydrogenase, and 3-hydroxybutyrate dehydrogenase.

In some embodiments, the redox polymer in the coating is crosslinked.

In some embodiments, the polymer is crosslinked during the electrode coating process, e.g., by including a crosslinking agent in a solution comprising the enzyme and the redox polymer used to coat the electrode. Inclusion of a crosslinking agent in the coating solution used to coat the electrode can result in physical entrapment of the enzyme in the coating layer, preventing the enzyme from diffusing away from the electrode or redox polymer during use.

In some embodiments, the presently disclosed subject matter provides a sensor for detecting an analyte of interest, wherein the sensor comprises a working electrode coated with a coating layer comprising a redox polymer (e.g., a redox homo- or copolymer) and an enzyme. In some embodiments, the analyte of interest comprises a plurality of analytes of interest. In some embodiments the analyte of interest is glucose. Thus, in some embodiments, the sensor is provided for glucose monitoring (e.g., continuous glucose monitoring). In some embodiments, the analyte of interest is ketone (e.g., 3-hydroxybutyrate). In some embodiments, the sensor is configured so that the electrode can be placed in fluid contact with interstitial fluid of a subject, e.g., a subject having diabetes or suspected of having diabetes. Thus, in some embodiments, the sensor is configured to be applied transdermally or subcutaneously to a subject (e.g., a diabetic patient).

In some embodiments, the working electrode coated with a coating layer comprising a redox polymer (e.g., a redox homo- or copolymer) and an enzyme can be further coated with an outer membrane (i.e., a membrane that covers the outer surface of the coating layer). The outer membrane can be a semi-permeable (e.g., amphiphilic or at least partially hydrophobic, so as to restrict the passage of water through the membrane) membrane. In some embodiments, the semi-permeable membrane is a polyurethane or silicone-based membrane. The addition of an outer membrane can increase the sensor lifetime, e.g., by restricting the amount of blood or other biological fluid which can interact with the enzyme. The enzyme consumption determines the stability and the lifetime of the sensors.

In some embodiments, the sensor comprises one or more additional electrodes. In some embodiments, the sensor further comprises a counter electrode and/or a reference electrode. The reference electrode can be an electrode electronically coupled to the working electrode to provide a stable reference voltage to the working electrode. The reference electrode can be an Ag/AgCl electrode or a metal oxide electrode (e.g., Pt oxide, Ir oxide, etc.).

The counter electrode can be an electrode that applies a voltage to the working electrode without being involved in electrochemical reactions like oxidation and reduction. The counter electrode can be a carbon electrode, a Pt electrode, a reduced graphene oxide electrode, a graphene oxide electrode or another stable surface. The sensor can further comprise a housing configured to house the electrodes while providing fluid communication with a biological sample.

In some embodiments, the sensor can be provided as part of a sensor system that further comprises an external transceiver (e.g., a transceiver configured the to power and/or communicate with the sensor when implanted in a subject and communicate with a reader) and a reader (e.g., to receive analyte sensing data from the transceiver and provide a readout of the data or to send the data to the cloud or an electronic device (e.g., a smart phone)). In some embodiments, the sensor system further comprises cloud storage & processing to provide feedback to the user and to their caregiver/doctor. The system can thereby provide information such as an analyte concentration, in a tissue (e.g., in an organ, vessel or fluids surrounding tissues and organs).

In some embodiments, the presently disclosed subject matter provides a method of sensing an analyte of interest, the method comprising: (a) applying a sample to a working electrode as described herein, and (b) measuring current to provide an output signal indicative of the presence or absence of the analyte. The measuring of step (b) can be performed by any suitable technique, e.g., cyclic voltammetry, potentiometry, amperometry, pulsed amperometry, differential pulse, or another electrochemical technique.

In some embodiments, the analyte of interest is selected from glucose, ketone, alcohol (e.g., ethanol), and a lactate. In some embodiments, the sample is a biological sample (e.g., suspected of containing the analyte of interest), such as, but not limited to, a blood sample, a saliva sample, or interstitial fluid. In some embodiments, the working electrode is present in a sensor further comprising a counter electrode and/or a working electrode.

In some embodiments, the analyte of interest is a ketone. Thus, in some embodiments, the working electrode is coated with a coating comprising a redox polymer and an enzyme that catalyzes a reaction of a ketone. For example, in some embodiments, the coating comprises 3-hydroxybutyrate dehydrogenase. In some embodiments, the method provides for continuous ketone monitoring (e.g., by taking ketone concentration measurements automatically every few seconds or minutes for one or more hours, days, or weeks). In some embodiments, the coating comprises a homopolymer of formula (II-1). In some embodiments, the coating comprises a copolymer of formula (III).

In some embodiments, the analyte of interest is glucose. Thus, in some embodiments, the working electrode is coated with a coating comprising a redox polymer and an enzyme that catalyzes a reaction of glucose, e.g., glucose oxidase or glucose dehydrogenase. In some embodiments, the coating comprises glucose dehydrogenase. In some embodiments, the method provides for continuous glucose monitoring (e.g., by taking glucose concentration measurements automatically every few seconds or minutes for one or more hours, days, or weeks). In some embodiments, the coating comprises a homopolymer of formula (II-1). In some embodiments, the coating comprises a copolymer of formula (III).

In some embodiments, the presently disclosed subject matter provides a method of preparing an electrode comprising a redox polymer coating. The method can comprise, for example, (a) dissolving the redox polymer (e.g., the redox homo- or copolymer) in an aqueous solution; (b) adding an enzyme (e.g., glucose oxidase, glucose dehydrogenase, or 3-hydroxybutyrate dehydrogenase) to the polymer solution; (c) coating the polymer solution on an electrode, to provide a coated electrode; and (d) drying the coated electrode. In some embodiments, the method further comprises adding a cross-linking agent to the polymer solution prior to step (c). The cross-linking agent can be selected from the group consisting of, but not limited to, glutaraldehyde, glyoxal, ethyl carbodiimide hydrochloride, aziridine, polyethyleneimine, trimethylolpropanetriacrylate, and pentaerythritol glycidyl ether.

In some embodiments, the polymer solution used in the coating of step (c) comprises about 10 wt % to about 50 wt % of the redox polymer. The coating of step (c) can be preformed by any suitable technique, e.g., dip coating, spin coating, spray coating, etc. In some embodiments, the drying can be performed in an oven (e.g., at about 55° C.) for a period of time. In some embodiments, the period of time is about 3 hours to about 16 hours.

In some embodiments, the presently disclosed subject matter provides a compound having a structure of formula (I′) (i.e., a redox mediator monomer):

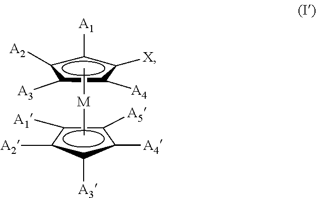

wherein M is a metal atom (e.g., selected from the group comprising Fe, Ru, Mn, Os, V, Co, Sc, Ti, Cr, Cu, Zn, Ni, Mo, Rh, Pd, Cd, Pt, and Ir); X is substituent that comprise a polymerizable functional moiety; A₁, A₂, A₃, and A₄ are each independently H or E, subject to the proviso that at least one of A₁, A₂, A₃, and A₄ is E; A₁′, A₂′, A₃′, A₄′, and A₅′ are each independently H or E, wherein at least two of A₁′, A₂′, A₃′, A₄′, and A₅′ are E; and E is an electron-donating aryl group substituent. In some embodiments, M is an Fe atom. In some embodiments, X is a substitutent that comprises one or more reactive functional groups selected from vinyl, alkenyl, OH, amino, epoxy, carboxylic acid, and isocyanate. In some embodiments, each E is independently selected from the group comprising alkyl, alkenyl, alkynyl, aralkyl, aryl, —N(R)₂, —NHR, —NH₂, —OH, —OR, —NHC(═O)R, and —OC(═O)R, wherein each R is independently selected from alkyl, aralkyl, and aryl (e.g., unsubstituted alkyl, aralkyl, and aryl). In some embodiments, at least three of A₁, A₂, A₃, and A₄ are E and at least four of A₁′, A₂′, A₃′, A₄′, and A₅′ are E. In some embodiments, each of A₁, A₂, A₃, A₄, A₁′, A₂′, A₃′, A₄′, and A₅′ is E. In some embodiments, each E is C1-C10 alkyl. In some embodiments, each E is methyl. In some embodiments, the compound of formula (I′) is one of compounds 4, 6, 8, 11, or 17 as described hereinabove.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example 1

### Synthesis of Redox Copolymer

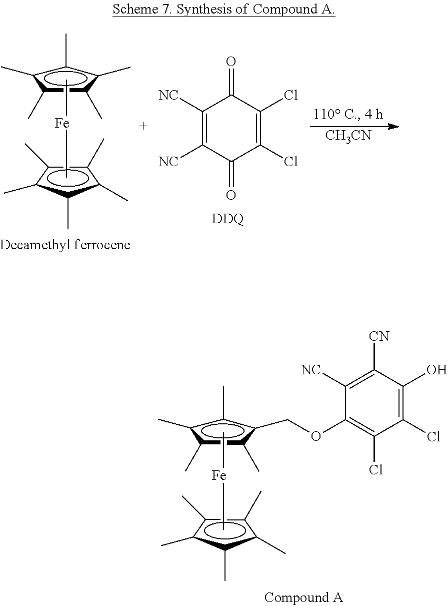

As shown hereinabove in Scheme 7, decamethyl ferrocene (1 eq), 2,3-dichloro-5,6-dicyano-1,4-benzoquinone (DDQ) (1 eq) and acetonitrile (10 parts with respect to (w.r.t) decamethyl ferrocene) were charged in a round bottom (RB) flask equipped with water condenser. The reaction was carried out at 110° C. for 4 hours. After completion of reaction, acetonitrile was removed using a rotary evaporator to provide Compound A.

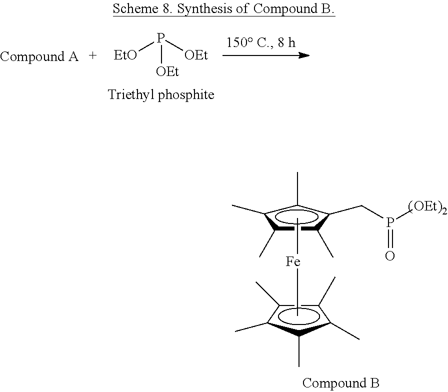

Next, in a second step as shown above in Scheme 8, compound A (1 eq) and triethylphosphite (1 eq) were placed in a reaction flask and the reaction was carried out at 150° C. for 18 hours, to provide Compound B.

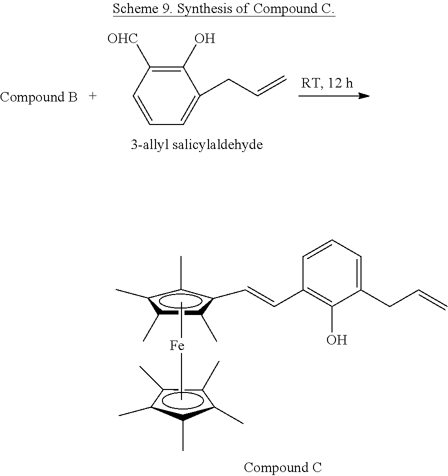

In a third step, as shown above in Scheme 9, compound B (1 eq), 3-allyl salicylaldehyde (1 eq), potassium tert-butoxide (10 μmol %) and dry tetrahydrofuran (THF, 10 parts w.r.t compound B) were placed in a reaction flask. The reaction was continued for 12 hours at room temperature. After completion of the reaction, the crude product was filtered and washed multiple times with THF, to provide Compound C, an exemplary redox mediator-containing monomer.

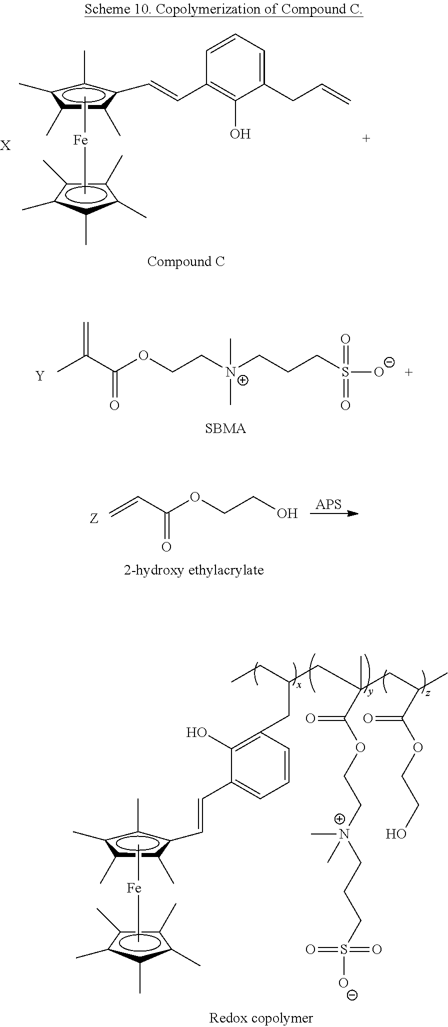

Compound C was copolymerized with a zwitterionic monomer (e.g., sulfobetainmethacrylate (SBMA)) and a water-soluble acrylate monomer (e.g., 2-hydroxyethyl acrylate (HEA)) to provide a redox copolymer. According to a general copolymerization procedure, a monomer mixture containing SBMA and HEA in a weight ratio of 9:1 to 1:9, and further comprising 1 wt % to 50 wt % Compound C (w.r.t. the total weight of SBMA and HEA) was added to a RB flask with ammonium persulfate (APS; 0.1 wt % to 10 wt %) and distilled water (10 parts, w.r.t. SBMA and HEA), and heated for a period of time.

In an exemplary copolymerization, a mixture comprising a weight ratio of 3:1 SBMA:HEA and further comprising compound C (5 wt % w.r.t the total weight of SBMA and HEA), ammonium persulfate (APS, 0.1 to 10 wt % (e.g., 4 wt %)) and distilled water (10 part, w.r.t total weight of SBMA and HEA) was mixed at 65° C. for 4 hours.

### Example 2

### Synthesis of Redox Homopolymer

A redox mediator monomer was prepared in three steps (i.e., as shown in Scheme 5, above). First, salicylaldehyde (1 eq) and epichlorohydrin (1 eq) were taken in the reaction flask and 40% NaOH (prepared using isopropyl alcohol) was slowly added in the reaction flask. After addition of NaOH, the reaction was continued for 4 hours at 110° C. The organic phase was separated and washed with distilled water multiple times until it reached neutral pH. The obtained compound was 2-(oxiran-2-ylmethoxy) benzaldehyde.

In a second step (2-(oxiran-2-ylmethoxy) benzaldehyde (1 eq), but-3-en-1-amine (1 eq) and solvent (10 parts w.r.t 2-(oxiran-2-ylmethoxy) benzaldehyde and but-3-en-1-amine) were mixed. The reaction was carried out at 60° C. for 5 hours. The obtained product was 2-(3-(but-3-en-1-ylamino)-2-hydroxypropoxy)benzaldehyde.

In a third step, 2-(3-(but-3-en-1-ylamino)-2-hydroxy-propoxy)benzaldehyde (1 eq) compound B (from Scheme 8 in Example 1, above; 1 eq) potassium tert-butoxide (10 μmol %) and dry tetrahydrofuran (THF; parts w.r.t compound B) were taken in a reaction flask. The reaction was continued for 12 hours at room temperature (RT). After completion of reaction the product was filtered and washed multiple times with THF. The obtained final product was compound 17 of Scheme 5.

To prepare the homopolymer, compound 17 (100 wt %) and ammonium persulfate (4 wt %) and distilled water (10 part (w.r.t compound 17) was taken in the reaction flask and the polymerization reaction was carried out at 65° C. for 4 hours.

### Example 3

Electrode Coating with Redox Polymer and Ketone Enzyme Coating

A polymer coating solution was prepared by dissolving 1 gram (20 wt %) of the redox mediator copolymer from Example 1 in 5 μmL of 0.1 M phosphate buffer solution (pH 7.4). The amount of redox mediator copolymer in the coating solution can be adjusted to increase the viscosity of the solution as desired between about 10 wt % and about 50 wt %. Once the polymer was dissolved, 100 μmg of 3-hydroxybutyrate dehydrogenase was added into the polymer solution and mixed. Then, 40 μL of glutaraldehyde was added to the resulting redox mediator copolymer/enzyme solution and mixed for 5 μminutes. After the mixing, the solution was dip coated onto a Pt working wire, using the following parameters:

### Dip:

Initial Speed: 350 μmm/min
Final Speed: 350 μmm/min

### Lift Speed:

Initial Speed: 300 μmm/min
Final Speed: 300 μmm/min

### General Parameters:

soaking time: 30 sec-1 μmin
Dry time: 3 hrs to 16 hrs (in an oven at 55° C.)
   The thickness and uniformity of the coating can be varied depending on the dipping parameters. Target thickness of coating varies from about 1 μm to about 20 μm, e.g., about 2 μm to about 5 μm.

### Example 4

### Ketone Sensing

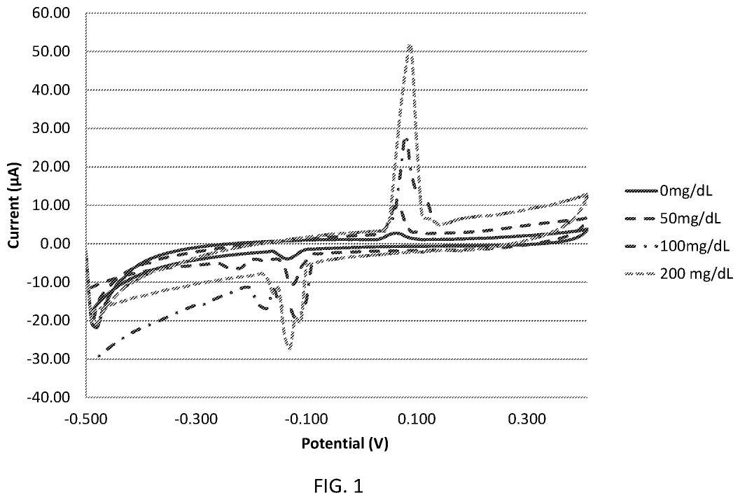

Pt wire electrodes coated with redox polymer and immobilized enzyme prepared in Example 3 were tested using a three-electrode system. The three-electrode system included a working electrode, a reference electrode and a counter electrode. The reference electrode was Ag/AgCl and the counter electrode was a platinum wire electrode. In a typical testing procedure, all three electrodes were placed in 20 μmL of a 0.1 μmolar (M) phosphate buffer solution (pH 7.4). A baseline cyclic voltammetry (CV) scan was conducted followed by addition of 3-hydroxybutyrate (HB) into the buffer at a concentration ranging from Omg/dL to 200 mg/dL. As shown in FIG. 1, the oxidation and reduction peaks increase as the HB concentration increases. The decamethylferrocene redox copolymer facilitated faster electron transfer to the electrode wires due to HB reaction with the 3-hydroxybutyrate dehydrogenase enzyme.

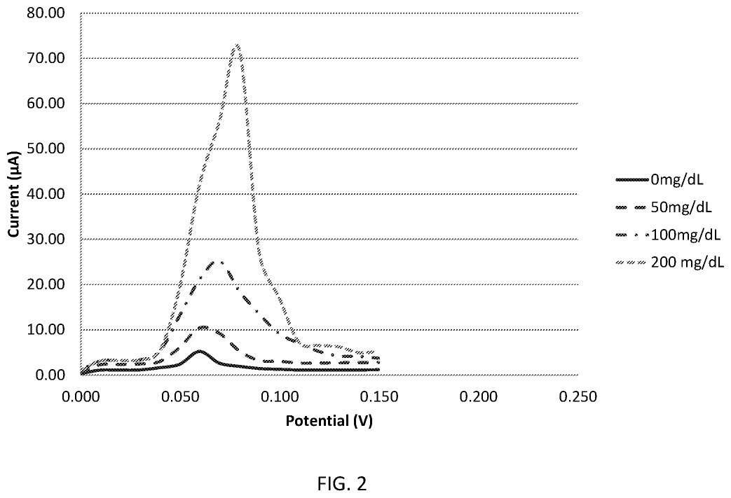

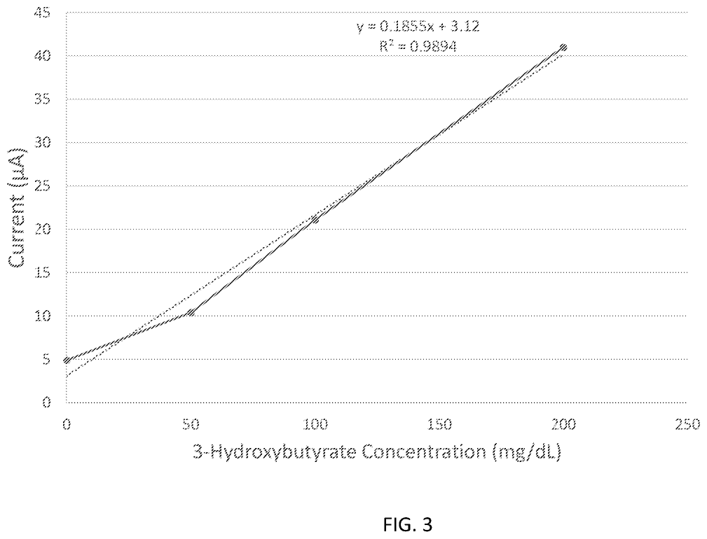

Differential pulse voltammetry (DPV) (see FIG. 2) was conducted on another coated sensor wire soaked in 0.1 M phosphate buffer (pH 7.4) with varying concentrations of HB (0 to 200 mg/dL). FIG. 3 shows the sensor response from FIG. 2 at a potential of 0.06 V vs. Ag/AgCl electrode.

### Example 5

### Glucose Enzyme Coating on the Working Electrode

A solution comprising 1 g (20 wt %) of redox mediator copolymer from Example 1 dissolved in 5 μmL of 0.1 M phosphate buffer solution (pH 7.4) was prepared. The amount of redox mediator copolymer can be adjusted to increase the viscosity of the solution (e.g., between about 10 wt % and about 50 wt %). Once the polymer was dissolved, 100 μmg of glucose dehydrogenase was added into the polymer solution and mixed. Then, 40 μL of glutaraldehyde was added to the redox mediator polymer/enzyme solution and mixed for 5 μmins.

After 5 μmins, the solution was dip coated onto a Pt working wire using the following parameters:

### Dip:

Initial Speed: 350 μmm/min
Final Speed: 350 μmm/min

### Lift Speed:

Initial Speed: 300 μmm/min
Final Speed: 300 μmm/min

### General Parameters:

soaking time: 30 sec-1 μmin
Dry time: 3 hrs to 16 hrs (in an oven at 55° C.)

The thickness and uniformity of the coating can be varied depending on the dipping parameters. Target thickness of the coating varies from about 1 μm to about 20 μm (e.g., about 2 μm to about 5 μm).

### Example 6

### Glucose Sensing

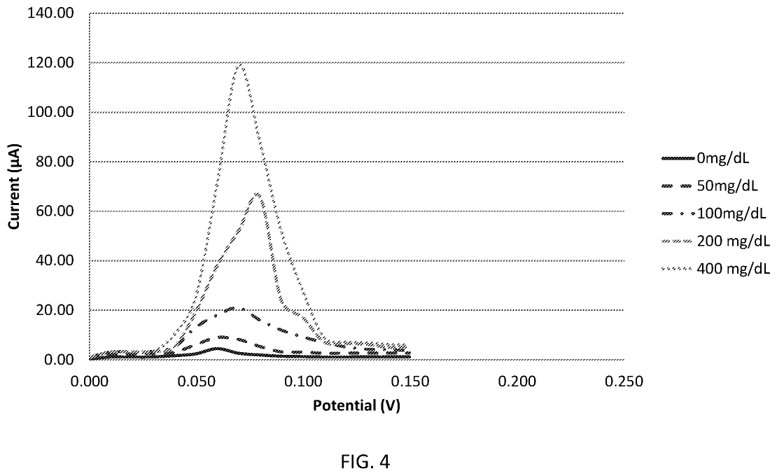

The same three electrode system as described in Example 4 was used for glucose sensing, excepting that the working electrode was a working electrode prepared as in Example 5, with glucose dehydrogenase as the enzyme immobilized in the polymer coating on the electrode. All three electrodes were placed in 20 μmL 0.1 M phosphate buffer solution (pH 7.4). A DPV scans were conducted by addition of D-glucose into the buffer to provide glucose concentrations between 0 mg/dL to 400 mg/dL. See FIG. 4. FIG. shows the sensor response at a potential of 0.06 V vs. Ag/AgCl electrode.

It will be understood that various details of the presently disclosed subject matter can be changed without departing from the scope of the presently disclosed subject matter. Furthermore, the foregoing description is for the purpose of illustration only, and not for the purpose of limitation.

What is claimed is:

1. A redox polymer comprising:
at least one polymeric chain; and
a plurality of metallocene groups, wherein each of the plurality of metallocene groups comprises a metal atom bound to two arene groups, wherein the two arene groups are together substituted by at least three electron-donating substituents and one substituent that is covalently or non-covalently bound to the at least one polymeric chain.

2. The redox polymer of claim 1, wherein each of the two arene groups is a cyclopentadienyl group.

3. The redox polymer of claim 1, wherein the metal atom is an atom of an element selected from the group consisting of Fe, Ru, Mn, Os, V, Co, Sc, Ti, Cr, Cu, Zn, Ni, Mo, Rh, Pd, Cd, Pt, and Ir, optionally wherein M is a Fe atom.

4. The redox polymer of claim 1, wherein the redox potential of each of the plurality of metallocene groups is less than about 0.2 volts (V) versus a silver/silver chloride (Ag/AgCl) reference electrode, optionally wherein the redox potential of each of the plurality of metallocene groups is less than about 0.1 V versus a Ag/AgCl reference electrode.

5. The redox polymer of claim 1, wherein each of the plurality of metallocene groups has a structure of formula (I): 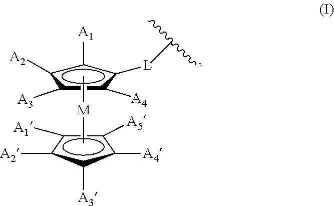
wherein:
M is a metal atom of an element selected from the group consisting of Fe, Ru, Mn, Os, V, Co, Sc, Ti, Cr, Cu, Zn, Ni, Mo, Rh, Pd, Cd, Pt, and Ir, optionally Fe;
L is a linking group that is covalently bonded to the at least one polymeric chain;
A₁, A₂, A₃, and A₄ are each independently H or E, subject to the proviso that at least one of A₁, A₂, A₃, and A₄ is E;
A₁′, A₂′, A₃′, A₄′, and A₅′ are each independently H or E, wherein at least two of A₁′, A₂′, A₃′, A₄′, and A₅′ are E; and
each E is an electron-donating substituent.

6. The redox polymer of claim 5, wherein each E is independently selected from the group consisting of alkyl, alkenyl, alkynyl, aralkyl, aryl, —N(R)₂, —NHR, —NH₂, —OH, —OR, —NHC(═O)R, and —OC(═O)R, wherein each R is independently selected from alkyl, aralkyl, and aryl.

7. The redox polymer of claim 6, wherein at least three of A₁, A₂, A₃, and A₄ are E and at least four of A₁′, A₂′, A₃′, A₄′, and A₅′ are E.

8. The redox polymer of claim 7, wherein each of A₁, A₂, A₃, A₄, A₁′, A₂′, A₃′, A₄′, and A₅′ is E.

9. The redox polymer of claim 5, wherein each E is C1-C10 alkyl.

10. The redox polymer of claim 10, wherein each E is methyl.

11. The redox polymer of claim 5, wherein L is selected from the group consisting of:

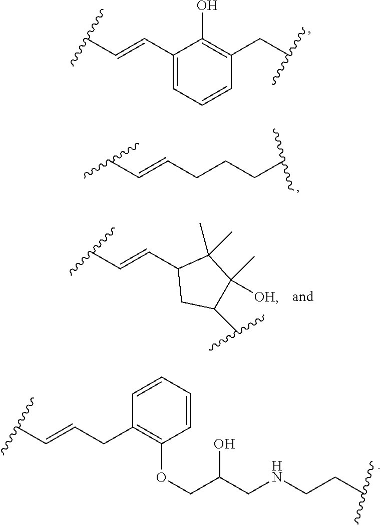

12. The redox polymer of claim 5, wherein the redox polymer is a homopolymer, optionally a homopolymer prepared by polymerization of a metallocene-containing monomer having a structure of formula (I′): 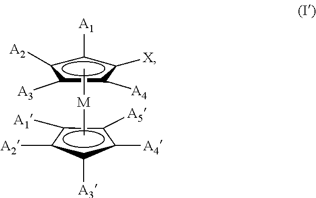
wherein:
M is an atom of an element selected from the group consisting of Fe, Ru, Mn, Os, V, Co, Sc, Ti, Cr, Cu, Zn, Ni, Mo, Rh, Pd, Cd, Pt, and Ir, optionally Fe;
X is a substituent that comprises one or more reactive functional groups selected from the group consisting of vinyl, alkenyl, OH, amino, epoxy, carboxylic acid, and isocyanate;
A₁, A₂, A₃, and A₄ are each independently H or E, subject to the proviso that at least one of A₁, A₂, A₃, and A₄ is E;
A₁′, A₂′, A₃′, A₄′, and A₅′ are each independently H or E, wherein at least two of A₁′, A₂′, A₃′, A₄′, and A₅′ are E; and
each E is an electron-donating aryl group substituent.

13. The redox polymer of claim 12, wherein the redox polymer has a structure of the formula: 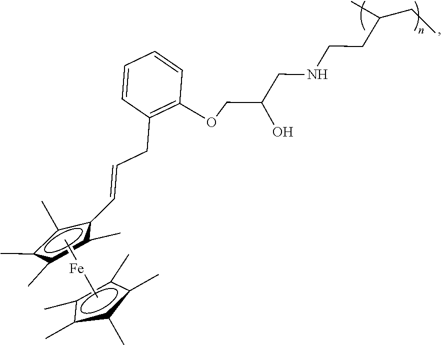
where n is an integer greater than 1.

14. The redox polymer of claim 5, wherein the redox polymer is a copolymer prepared by copolymerization of at least two different monomers, wherein one of the monomers is a metallocene-containing monomer having a structure of formula (I′): 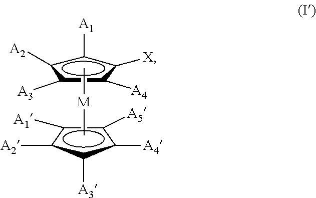
wherein:
M is an atom of an element selected from the group consisting of Fe, Ru, Mn, Os, V, Co, Sc, Ti, Cr, Cu, Zn, Ni, Mo, Rh, Pd, Cd, Pt, and Ir, optionally Fe;
X is a substituent that comprises one or more reactive functional groups selected from the group consisting of vinyl, alkenyl, OH, amino, epoxy, carboxylic acid, and isocyanate;
A₁, A₂, A₃, and A₄ are each independently H or E, subject to the proviso that at least one of A₁, A₂, A₃, and A₄ is E;
A₁′, A₂′, A₃′, A₄′, and A₅′ are each independently H or E, wherein at least two of A₁′, A₂′, A₃′, A₄′, and A₅′ are E; and
E is an electron-donating aryl group substituent;
optionally wherein the copolymer is a random copolymer, a block copolymer and a graft copolymer.

15. The redox polymer of claim 14, wherein at least one of the at least two different monomers is a water-soluble acrylic monomer or a zwitterionic monomer.

16. The redox polymer of claim 15, wherein the copolymer is a terpolymer prepared by copolymerization of (i) a metallocene-containing monomer having a structure of formula (I′), (ii) a zwitterionic monomer, and (iii) a third monomer, optionally a water soluble acrylic monomer.

17. The redox polymer of claim 16, wherein the copolymer has a structure of formula (III): 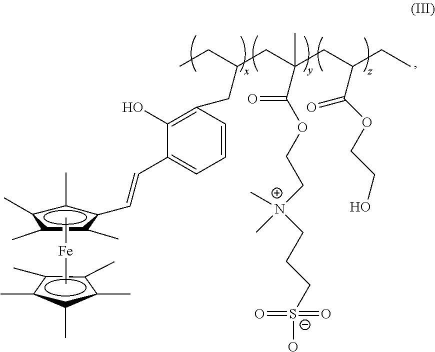
where x, y, and z are each integers, optionally wherein each of x, y, and z are integers greater than 1, further optionally wherein each of x, y, and z are integers greater than 10.

18. A blend comprising the redox polymer of claim 1 and one or more additional polymers, optionally wherein the one or more additional polymers are water soluble.

19. An electrode coated with a coating comprising the redox polymer of claim 1 or a blend thereof.

20. The electrode of claim 19, wherein the coating has a thickness of about 1 μmicrometer (μm) to about 20 μm, optionally about 2 μm to about 5 μm.

21. The electrode of claim 19, wherein the coating further comprises an enzyme, optionally an oxidase or a dehydrogenase, further optionally wherein the enzyme is selected from glucose oxidase, glucose dehydrogenase, and 3-hydroxybutyrate dehydrogenase.

22. The electrode of claim 21, wherein the redox polymer is crosslinked.

23. The electrode of claim 21, further comprising an outer membrane over the coating, wherein the outer membrane is a semi-permable membrane, optionally wherein the semi-permeable membrane is a polyurethane or silicone-based membrane.

24. A sensor for detecting an analyte of interest, wherein the sensor comprises a working electrode, wherein the working electrode is an electrode of claim 21, optionally wherein the analyte of interest comprises a plurality of analytes of interest.

25. The sensor of claim 24, wherein the sensor further comprises a counter electrode and/or a reference electrode.

26. A method of sensing an analyte of interest, the method comprising:
(a) applying a sample to a working electrode of a sensor of claim 24, optionally wherein the sample comprises a sample suspected of comprising the analyte; and
(b) measuring current to provide an output signal indicative of the presence or absence of the analyte.

27. The method of claim 26, wherein the sample is a biological sample, optionally a blood sample or interstitial fluid.

28. The method of claim 26, wherein the analyte of interest is selected from glucose, a ketone, an alcohol, and a lactate.

29. The method of claim 26, wherein the analyte of interest is a ketone and the working electrode is coated with a coating comprising 3-hydroxybutyrate dehydrogenase.

30. The method of claim 26, wherein the analyte of interest is glucose and the working electrode is coated with a coating comprising glucose dehydrogenase.

31. A method of preparing an electrode of claim 19, the method comprising:
(a) dissolving the redox polymer or blend in an aqueous solution to provide a polymer solution;
(b) adding an enzyme to the polymer solution;
(c) coating the polymer solution onto an electrode, to provide a coated electrode; and
(d) drying the coated electrode.

32. The method of claim 31, wherein the method further comprises adding a cross-linking agent to the polymer solution prior to step (c), optionally wherein the cross-linking agent is selected from the group consisting of glutaraldehyde, glyoxal, ethyl carbodiimide hydrochloride, aziridine, polyethyleneimine, trimethylolpropanetriacrylate, and pentaerythritol glycidyl ether.

33. A compound having a structure of formula (I′): 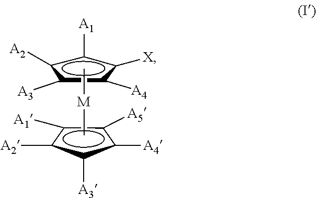
wherein:
M is an atom of a metal element selected from the group consisting of Fe, Ru, Mn, Os, V, Co, Sc, Ti, Cr, Cu, Zn, Ni, Mo, Rh, Pd, Cd, Pt, and Ir, optionally Fe;
X is a substituent that comprises one or more reactive functional groups selected from the group consisting of vinyl, alkenyl, OH, amino, epoxy, carboxylic acid, and isocyanate;
A₁, A₂, A₃, and A₄ are each independently H or E, subject to the proviso that at least one of A₁, A₂, A₃, and A₄ is E;
A₁′, A₂′, A₃′, A₄′, and A₅′ are each independently H or E, wherein at least two of A₁′, A₂′, A₃′, A₄′, and A₅′ are E; and
E is an electron-donating aryl group substituent.

34. The compound of claim 33, wherein each E is independently selected from the group consisting of alkyl, alkenyl, alkynyl, aralkyl, aryl, —N(R)₂, —NHR, —NH₂, —OH, —OR, —NHC(═O)R, and O—C(═O)R, wherein each R is independently selected from alkyl, aralkyl, and aryl.

35. The compound of claim 33, wherein at least three of A₁, A₂, A₃, and A₄ are E and at least four of A₁′, A₂′, A₃′, A₄′, and A₅′ are E.

36. The compound of claim 35, wherein each of A₁, A₂, A₃, A₄, A₁′, A₂′, A₃′, A₄′, and A₅′ is E.

37. The compound of claim 33, wherein each E is C1-C10 alkyl.

38. The compound of claim 37, wherein each E is methyl.
